(19) United States
(12) Patent Application Publication
(10) Pub. No.: US 2024/0097499 A1
(43) Pub. Date: Mar. 21, 2024

(54) DIRECTIONAL CONTROL OF MULTI-COIL ARRAY FOR APPLICATIONS IN RECHARGE SYSTEMS

(71) Applicant: Medtronic, Inc., Minneapolis, MN (US)

(72) Inventors: Andrew Thomas Fried, St. Paul, MN (US); Venkat R. Gaddam, Plymouth, MN (US); Brett Otteson, Minneapolis, MN (US); Robert J. Monson, St. Paul, MN (US); Ashutosh Mehra, St. Paul, MN (US); Jacob P. Komarek, Fridley, MN (US); Sandra L. Hawley, Oceanside, CA (US); Derek J. Olmschenk, Lino Lakes, MN (US); Jacob L. Hammond, Roseville, MN (US)

(21) Appl. No.: 18/263,170

(22) PCT Filed: Feb. 24, 2022
(86) PCT No.: PCT/US2022/017635

§ 371 Date: Jul. 27, 2023

Related U.S. Application Data
(60) Provisional application No. 63/153,237, filed on Feb. 24, 2021.

(51) Int. Cl. H02J 50/40; H02J 50/00; H02J 50/10; H02J 50/80

(52) U.S. Cl. CPC H02J 50/40; H02J 50/005; H02J 50/10; H02J 50/80

(57) ABSTRACT

A system that includes a power transmitting antenna (124) with a coiled conductor defined by a first axis and a second axis perpendicular to the first axis, where a single plane comprises the first axis and the second axis. The system includes a support layer (140, 142) comprising: a substantially planar top surface and a substantially planar bottom surface opposite the substantially planar top surface arranged parallel to the plane. The support layer also comprises a material with a predetermined resiliency. The support layer is configured to support a mass of a user and maintain a predetermined spacing between the plane of the power transmitting antenna and the user during compression of the material from the mass of the user.

This application is a PCT application that claims priority to U.S. Provisional Patent Application No. 63/153,237, filed Feb. 24, 2021, the entire contents of which is incorporated herein by reference.

### TECHNICAL FIELD

The disclosure relates to wireless power transfer systems.

### BACKGROUND

Portable electronic devices may be located where providing electrical power via a wired connection to a power source may be difficult. Some portable electronic devices may include a power supply such as a rechargeable battery or some other electrical energy storage device. In other examples, portable electronic devices may not include an internal power supply and instead be configured to directly receive wireless power to operate. Power may be transmitted wirelessly from a power transmitting unit (PTU) to a power receiving unit (PRU) for example by transmitting radio frequency (RF) energy, by inductive coupling and so on. In some examples the PTU and PRU may also communicate, e.g., send digital messages back and forth, using radio frequency (RF) communication or inductive communication before, during or after transferring power. In some examples, the PTU may wirelessly transfer energy to the PRU to recharge, for example, a battery, a storage capacitor or some other electrical energy storage device in the PRU. Some examples of portable electronic devices may include implantable medical devices. Implantable medical devices may receive wireless power via a transcutaneous power transfer configured to either directly power the device or to recharge the electrical energy storage device.

### SUMMARY

In general, the disclosure describes devices, systems, and techniques related to providing power transfer from a power transmitting unit to power receiving unit. In the example of recharging an electrical energy storage device, such as a battery, consistent power transfer may result in consistent recharge durations.

In one example, this disclosure describes a system comprising a power transmitting antenna comprising a coiled conductor defined by a first axis and a second axis perpendicular to the first axis, wherein a single plane comprises the first axis and the second axis; a support layer comprising: a substantially planar top surface and a substantially planar bottom surface opposite the substantially planar top surface arranged parallel to the plane; and a material with a predetermined resiliency; wherein the support layer is configured to support a mass of a user and maintain a predetermined spacing between the plane of the power transmitting antenna and the user during compression of the material from the mass of the user.

In another example, this disclosure describes device that includes a power transmitting antenna comprising a first coiled conductor defined by a first axis and a second axis perpendicular to the first axis, the first axis and the second axis defining a first plane; and a second coiled conductor defined by a third axis and a fourth axis perpendicular to the third axis, the third axis and the fourth axis defining a second plane; and drive circuitry configured to: generate a first magnetic field in the first coiled conductor with a first drive signal; generate a second magnetic field in the second coiled conductor with a second drive signal, wherein the second drive signal has a phase difference from the first drive signal and an amplitude difference from the first drive signal; and adjust an angle of a combined magnetic field comprising the first electromagnetic field and the second magnetic field based on the phase difference and the amplitude difference, wherein the angle is relative to the first plane.

In another example, this disclosure describes a system comprising a power transmitting antenna includes a first coiled conductor defined by a first axis and a second axis perpendicular to the first axis, the first axis and the second axis defining a first plane; and a second coiled conductor defined by a third axis and a fourth axis perpendicular to the third axis, the third axis and the fourth axis defining a second plane; a drive circuit configured to: generate a first magnetic field in the first coiled conductor with a first drive signal; generate a second magnetic field in the second coiled conductor with a second drive signal, wherein the second drive signal has a phase difference from the first drive signal and an amplitude difference from the first drive signal; and adjust an angle of a combined magnetic field comprising the first electromagnetic field and the second magnetic field based on the phase difference and the amplitude difference, wherein the angle is relative to the first plane; and processing circuitry operatively coupled to a memory and to the drive circuit, the processing circuitry configured to control the operation of the drive circuit.

In another example, this disclosure describes a power transmitting antenna the antenna comprising a coiled conductor defined by a first axis and a second axis perpendicular to the first axis, wherein the antenna defines an outside edge and an inside edge of a coil, wherein the outside edge defines an outside dimension and the inside edge defines an inside dimension; and wherein a single plane comprises the first axis and the second axis,

the coiled conductor comprising a first portion of the coiled conductor wrapped with a plurality of turns in a clockwise direction from the outside dimension to the inside dimension; a second portion of the coiled conductor wrapped with a plurality of turns in a counter-clockwise direction from the inside dimension to the outside dimension, wherein the first portion is electrically connected to the second portion.

In another example, this disclosure describes a method comprising controlling, by processing circuitry operatively coupled to a memory, a drive circuit to activate a first coil of a power transmitting antenna; receiving, by the processing circuitry, an indication from a power receiving unit of a first amount of power received from the power transmitting antenna; storing, by the processing circuitry at a memory location of the memory, a first value associated with the first amount of power; controlling, by the processing circuitry, the drive circuit to deactivate the first coil; controlling, by the processing circuitry, the drive circuit to activate a second coil of the power transmitting antenna; receiving, by the processing circuitry, an indication from the power receiving unit of a second amount of power received from the power transmitting antenna; storing, by the processing circuitry at a memory location of the memory, a second value associated with the second amount of power; controlling, by the processing circuitry, the drive circuit to deactivate the second coil; comparing, by the processing circuitry, the first value to the second value; and controlling, by the processing circuitry and based on the comparison and responsive to the first value being greater than the second value, the drive circuit to deliver power to the power receiving unit by activating the first coil.

The details of one or more examples are set forth in the accompanying drawings and the description below. Other features, objects, and advantages will be apparent from the description, drawings, and claims.

### BRIEF DESCRIPTION OF DRAWINGS

FIG. 1 is a conceptual diagram illustrating example system that includes an implantable medical device (IMD) and an external charging device configured to charge a rechargeable power source of the IMD via an energy transfer coil.

FIG. 2 is a block diagram illustrating example components of the IMD of FIG. 1.

FIG. 3 is a block diagram of an example external charging device of FIG. 1.

FIGS. 4A and 4B are conceptual diagrams illustrating example primary and secondary coils of a wireless power transfer system along with associated magnetic field and electric field.

FIG. 4C is a conceptual diagram illustrating an example multi-coil recharging pad.

FIG. 5 is a conceptual diagram illustrating an example remote control for example wireless power transfer systems of this disclosure.

FIG. 6 is a conceptual diagram illustrating an example magnetic field strength of a single coil.

FIG. 7 is a conceptual diagram illustrating an example primary coil of this disclosure.

FIGS. 8A and 8B are conceptual diagrams illustrating an example separation pad for the electric (E) and magnetic (H) field protection in recharge mattress pad according to one or more techniques of this disclosure.

FIGS. 9A, 9B, 9C, and 9D are diagrams illustrating various examples of the charging system of this disclosure.

FIG. 10A is a graph illustrating the impact on charge time from changes in the spacing between a primary coil and a secondary coil.

FIG. 10B is a graph illustrating an example battery charge current limit in the power receiving device.

FIGS. 11A, 11B, and 11C are conceptual diagrams illustrating simulated charge time contours for an implantable medical device being charged by a mattress pad based wireless power transfer system.

FIGS. 12A, 12B, and 12C are conceptual diagrams illustrating an example multiple coil wireless power transfer system configured to compensate for the relative orientation of the primary coil and the secondary coil.

FIGS. 13A, 13B and 13C are conceptual diagrams illustrating an example return-wrapping technique for transmitting coils in wireless power transfer applications.

FIGS. 13D, 13E, 13F, and 13G are conceptual diagrams illustrating the electromagnetic field generated by a transmitting coil according to one or more techniques of this disclosure.

FIGS. 14A, 14B and 14C are flowcharts illustrating an example operation of a system of this disclosure.

FIG. 15A is a schematic diagram illustrating a portion of the driving circuitry for an example wireless power transfer system.

FIG. 15B is a schematic diagram illustrating an example power supply for a wireless power transmitting system.

### DETAILED DESCRIPTION

This disclosure describes devices, systems, and techniques for wireless power transfer. Some of the examples include: (a) a return-wrapping technique for transmitting coils in wireless power transfer applications, (b) an algorithm for passive recharge system coil activation, (c) a separation pad for e/h field protection in a passive recharge mattress pad, and (d) directional control of a multi-coil array for applications in passive recharge systems. The above examples may be combined with each other in any combination or two or more techniques.

In the realm of rechargeable devices, including medical implants, it may be desirable to ease the recharging process by creating recharging systems that produce a relatively strong magnetic field over a large area so that the implant charges in a time-efficient manner and is not constrained to a tight position. One way to accomplish this is to use a physically large transmitting coil with many turns. However, this may result in a coil with a relatively large inductance, which when powered with an alternating current (AC) creates a relatively large voltage drop across its turns. On a traditionally wound coil, this difference in electrical potential from the inner-most turn to the outer-most turn of the coil may create an electric field that exceeds what is allowed by IEEE Std C95.1-2019 for persons in unrestricted environments.

A return-wound coil of this disclosure has turns that wrap inwards and then back outwards again. In this way, the voltage drop is divided in half across the inwardly-spiraling turns and the outwardly-spiraling turns, and the electric field produced by the inward half provides a canceling effect on the electric field produced by the outward half. In this manner the return-wound coil of this disclosure may provide a magnetic field over a large area for convenient power transfer, but the total electric field should be near-zero and within the forementioned regulations. In some examples, it may be desirable to include extra insulation between the two halves (inward and outward spirals) because of the high voltage potential difference between the first and last turn.

The following is an example algorithm for managing a passive recharge system. For a multi-coil wireless electrical energy transfer system may use the following algorithm with three stages: check communication, activate each coil independently and measure response from power receiving device, then activate most effective coil and begin active charging.

The primary coil for the power transmitting unit generates the electric (E) and magnetic (H) field to wirelessly transfer energy to the power receiving unit. In the example of a power receiving unit that is an implantable medical device, one or more primary coils may be placed in a mattress pad to provide convenient recharging while the patient is sleeping. Industry standards govern the maximum electromagnetic field which are allowed and considered safe. By separating a relatively large coil recharge system from the patient by a pad of, for example of a few centimeters thickness, the charging system may transfer wireless energy to a greater distance above the mattress. The greater distance comes from both the shape of the magnetic field and that the greater separation may allow for increased power to the coil, while remaining within safe energy levels. The size and shape of the primary coil may change the shape of the magnetic field, and therefore the effective energy transfer distance.

In some examples, a multi-coil array for applications in passive recharge systems may provide directional control of the electromagnetic field by exercising at least two coils simultaneously out of phase with each other. In some examples, the efficiency of wireless energy transfer may improve when the primary and secondary coils are approximately parallel, when compared to being at a relative angle to each other. In some examples, to exercise at least two coils simultaneously out of phase with each other, e.g., between 1-180 degrees out of phase so that the angle of the magnetic field may be adjusted. Both the amplitude and phase of the two coils may be adjusted to identify the field direction. In this manner, the patient could “roll on their side” or “lay at an angle” as long as they are laying vertically along the length of the bed. Thus, patient “roll” would be accounted for in such a passive recharge system, but patient “yaw” and “pitch” may not require adjustment. Adjusting the phase to account for patient roll angle may simplify the overall system design and provide efficient energy transfer. Additionally, instructions for use may indicate that the patient should be laying generally aligned with the long dimension of the bed for the system to charge with good results. This may have an advantage of reducing the stress on the patient, so they do not have to worry about laying “flat on their back”.

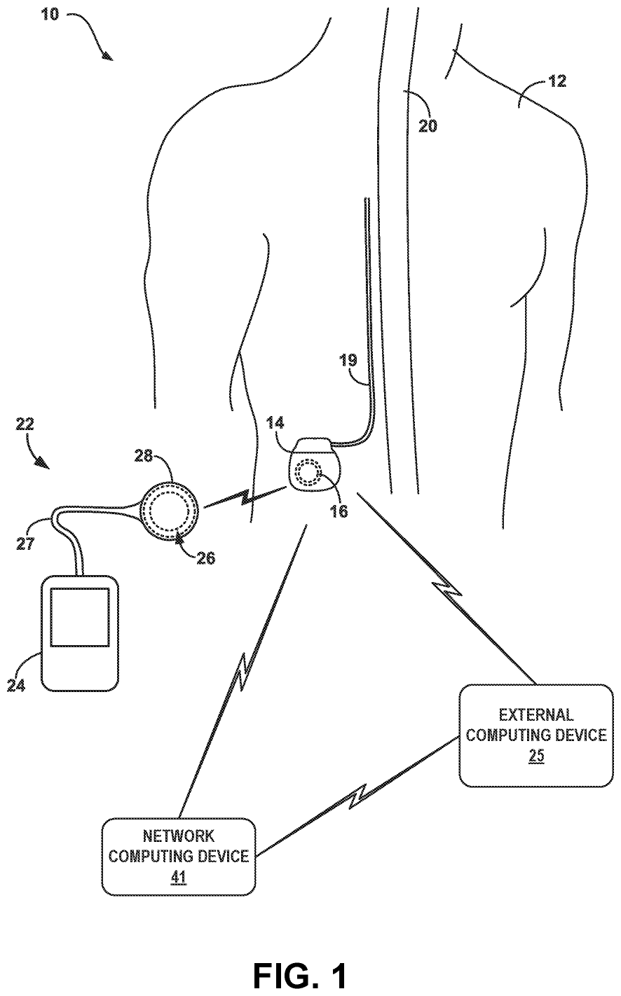

FIG. 1 is a conceptual diagram illustrating example system 10 that includes an implantable medical device (IMD) 14 and an external charging device 22 configured to charge a rechargeable power source of the IMD 14 via an energy transfer coil 26. Although the techniques described in this disclosure are generally applicable to a variety of medical devices including medical devices such as patient monitors, electrical stimulators, or drug delivery devices, application of such techniques to implantable neurostimulators will be described for purposes of illustration. More particularly, the disclosure will refer to an implantable neurostimulation (INS) system for use in spinal cord stimulation therapy, but without limitation as to other types of medical devices. An IMD configured for neurostimulation may be referred to as an INS in this disclosure.

As shown in FIG. 1, system 10 includes an IMD 14 and external charging device 22 shown in conjunction with a patient 12, who is ordinarily a human patient. In the example of FIG. 1, IMD 14 is an implantable electrical stimulator that delivers neurostimulation therapy to patient 12, e.g., for relief of chronic pain or other symptoms. Generally, IMD 14 may be a chronic electrical stimulator that remains implanted within patient 12 for weeks, months, or even years. In the example of FIG. 1, IMD 14 and lead 19 may be directed to delivering spinal cord stimulation therapy. In other examples, IMD 14 may be a temporary, or trial, stimulator used to screen or evaluate the efficacy of electrical stimulation for chronic therapy. IMD 14 may be implanted in a subcutaneous tissue pocket, within one or more layers of muscle, or other internal location. IMD 14 includes a rechargeable power source (not shown) and IMD 14 is coupled to lead 19.

Electrical stimulation energy, which may be constant current or constant voltage based pulses, for example, is delivered from IMD 14 to one or more targeted locations within patient 12 via one or more electrodes (not shown) of lead 19. The parameters for a program that controls delivery of stimulation energy by IMD 14 may include information identifying which electrodes have been selected for delivery of stimulation according to a stimulation program, the polarities of the selected electrodes, i.e., the electrode configuration for the program, and voltage or current amplitude, pulse rate, pulse shape, and pulse width of stimulation delivered by the electrodes. Electrical stimulation may be delivered in the form of stimulation pulses or continuous waveforms, for example. In other examples, IMD 14 may be configured to monitor patient biological signals, such as biological impedance, cardiac signals, temperature, activity, and so on. In some examples IMD 14 may not deliver stimulation therapy.

In the example of FIG. 1, lead 19 is disposed within patient 12, e.g., implanted within patient 12. Lead 19 tunnels through tissue of patient 12 from along spinal cord 20 to a subcutaneous tissue pocket or other internal location where IMD 14 is disposed. Although lead 19 may be a single lead, lead 19 may include a lead extension or other segments that may aid in implantation or positioning of lead 19. In addition, a proximal end of lead 19 may include a connector (not shown) that electrically couples to a header of IMD 14. Although only one lead 19 is shown in FIG. 1, system 10 may include two or more leads, each coupled to IMD 14 and directed to similar or different target tissue sites. For example, multiple leads may be disposed along spinal cord 20 or leads may be directed to spinal cord 20 and/or other locations within patient 12. Lead 19 may carry one or more electrodes that are placed adjacent to the target tissue, e.g., spinal cord 20 for spinal cord stimulation (SCS) therapy.

In alternative examples, lead 19 may be configured to deliver stimulation energy generated by IMD 14 to stimulate one or more sacral nerves of patient 12, e.g., sacral nerve stimulation (SNS). SNS may be used to treat patients suffering from any number of pelvic floor disorders such as pain, urinary incontinence, fecal incontinence, sexual dysfunction, or other disorders treatable by targeting one or more sacral nerves. Lead 19 and IMD 14 may also be configured to provide other types of electrical stimulation or drug therapy (e.g., with lead 19 configured as a catheter). For example, lead 19 may be configured to provide deep brain stimulation (DBS), peripheral nerve stimulation (PNS), or other deep tissue or superficial types of electrical stimulation. In other examples, lead 19 may provide one or more sensors configured to allow IMD 14 to monitor one or more parameters of patient 12. The one or more sensors may be provided in addition to, or in place of, therapy delivery by lead 19.

IMD 14 delivers electrical stimulation therapy to patient 12 via selected combinations of electrodes carried by lead 19. The target tissue for the electrical stimulation therapy may be any tissue affected by electrical stimulation energy, which may be in the form of electrical stimulation pulses or waveforms. In some examples, the target tissue includes nerves, smooth muscle, and skeletal muscle. In the example illustrated by FIG. 1, the target tissue for electrical stimulation delivered via lead 19 is tissue proximate spinal cord 20 (e.g., one or more target locations of the dorsal columns or one or more dorsal roots that branch form spinal cord 20. Lead 19 may be introduced into spinal cord 20 via any suitable region, such as the thoracic, cervical or lumbar regions. Stimulation of dorsal columns, dorsal roots, and/or peripheral nerves may, for example, prevent pain signals from traveling through spinal cord 20 and to the brain of the patient. Patient 12 may perceive the interruption of pain signals as a reduction in pain and, therefore, efficacious therapy results. For treatment of other disorders, lead 19 may be introduced at any exterior location of patient 12.

Although lead 19 is described as generally delivering or transmitting electrical stimulation signals, lead 19 may additionally or alternatively transmit electrical signals from patient 12 to IMD 14 for monitoring. For example, IMD 14 may utilize detected nerve impulses to diagnose the condition of patient 12 or adjust the delivered stimulation therapy. Lead 19 may thus transmit electrical signals to and from patient 12.

A user, such as a clinician or patient 12, may interact with a user interface of an external computing device 25 to communicate with and in some examples, to program IMD 14. Programming of IMD 14 may refer generally to the generation and transfer of commands, programs, or other information to control the operation of IMD 14. For example, the external programmer may transmit programs, parameter adjustments, program selections, group selections, or other information to control the operation of IMD 14, e.g., by wireless telemetry or wired connection.

In some cases, external computing device 25 may be characterized as a physician or clinician programmer if it is primarily intended for use by a clinician. In other cases, external computing device 25 may be characterized as a patient programmer if it is primarily intended for use by a patient. A patient programmer is generally accessible to patient 12 and, in many cases, may be a portable device that may accompany the patient throughout the patient's daily routine. In general, a physician or clinician programmer may support selection and generation of programs by a clinician for use by stimulator 14, whereas a patient programmer may support adjustment and selection of such programs by a patient during ordinary use. In other examples, external charging device 25 may be included, or part of, an external programmer. In this manner, a user may program and charge IMD 14 using one device, or multiple devices.

System 10 may also include network computing device 41, which may be implemented as a cloud server, a home or office network server, a mobile phone, tablet computer, laptop or other computing device. Functions attributed to processing circuitry in this disclosure may be performed by any one or shared across any combination of processing circuitry of system 10. Network computing device 41 may include a user interface configured to receive user input and display information to a user.

IMD 14 may be constructed of any polymer, metal, or composite material sufficient to house the components of IMD 14 within patient 12. In this example, IMD 14 may be constructed with a biocompatible housing, such as titanium or stainless steel, or a polymeric material such as silicone or polyurethane, and surgically implanted at a site in patient 12 near the pelvis, abdomen, or buttocks. The housing of IMD 14 may be configured to provide a hermetic seal for components, such as a rechargeable power source. In addition, the housing of IMD 14 may be selected of a material that facilitates receiving energy to charge a rechargeable power source.

As described herein, secondary coil 16 may be included within IMD 14. However, in other examples, secondary coil 16 could be located external to a housing of IMD 14, separately protected from fluids of patient 12, and electrically coupled to electrical components of IMD 14. This type of configuration of IMD 14 and secondary coil 16 may provide implant location flexibility when anatomical space available for implantable devices is minimal and/or improved inductive coupling between secondary coil 16 and primary coil 26. In any case, an electrical current may be induced within secondary coil 16 to charge the battery of IMD 14 when energy transfer coil 26 (e.g., a primary coil) produces a magnetic field that is aligned with secondary coil 16. The induced electrical current may first be conditioned and converted by a charging module (e.g., a charging circuit) to an electrical signal that can be applied to the battery with an appropriate charging current. For example, the inductive current may be an alternating current that is rectified to produce a direct current suitable for charging the battery. In some examples, primary coil 26 may comprise multiple separate coils that are displaced in location from each other.

The rechargeable power source of IMD 14 may include one or more capacitors, batteries, or components (e.g., chemical or electrical energy storage devices). Example batteries may include lithium-based batteries, nickel metal-hydride batteries, or other materials. The rechargeable power source may be replenished, refilled, or otherwise capable of increasing the amount of energy stored after energy has been depleted. The energy received from secondary coil 16 may be conditioned and/or transformed by a charging circuit. The charging circuit may then send an electrical signal used to charge the rechargeable power source when the power source is fully depleted or only partially depleted.

Charging device 22 may be used to recharge the rechargeable power source within IMD 14 implanted in patient 12. Charging device 22 may be a hand-held device, a portable device, or a stationary charging system. In any case, charging device 22 may include components necessary to charge IMD 14 through tissue of patient 12. Charging device 22 may include housing 24 and energy transfer coil 26. In addition, heat sink device 28 may be removably attached to energy transfer coil 26 to manage the temperature of then energy transfer coil during charging sessions. Housing 24 may enclose operational components such as a processor, memory, user interface, telemetry module, power source, and charging circuit configured to transmit energy to secondary coil 16 via energy transfer coil 26. Although a user may control the recharging process with a user interface of charging device 22, charging device 22 may alternatively be controlled by another device (e.g., an external programmer). In other examples, charging device 22 may be integrated with an external programmer, such as a patient programmer carried by patient 12.

Charging device 22 and IMD 14 may utilize any wireless power transfer techniques that are capable of recharging the power source of IMD 14 when IMD 14 is implanted within patient 12. In one example, system 10 may utilize inductive coupling between primary coils (e.g., energy transfer coil 26) and secondary coils (e.g., secondary coil 16) of charging device 22 and IMD 14. In inductive coupling, energy transfer coil 26 is placed near implanted IMD 14 such that energy transfer coil 26 is aligned with secondary coil 16 of IMD 14. Charging device 22 may then generate an electrical current in energy transfer coil 26 based on a selected power level for charging the rechargeable power source of IMD 14. When the primary and secondary coils are aligned, the electrical current in the primary coil may magnetically induce an electrical current in the secondary coil within IMD 14. Since the secondary coil is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between charging device 22 and IMD 14.

Energy transfer coil 26 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Energy transfer coil 26 may also include a housing that encases the coil. The housing may be constructed of a flexible material such that the housing promotes, or does not inhibit, flexibility of the coil. Energy transfer coil 26 may be external of housing 24 such that energy transfer coil 26 can be placed on the skin of patient 12 proximal to IMD 14. In this manner, energy transfer coil 26 may be tethered to housing 24 using cable 27 or other connector that may be between approximately a few inches and several feet in length. In other examples, energy transfer coil 26 may be disposed on the outside of housing 24 or even within housing 24. Energy transfer coil 26 may thus not be tethered to the housing 24 in other examples.

Heat sink device 28 may be removably attached to energy transfer coil 26. In examples where energy transfer coil 26 is disposed on or within housing 24, heat sink device 28 may be configured to be removably attached to housing 24.

Together, system 10 may include energy transfer coil 26 and heat sink device 28. Energy transfer coil 26 may be configured to recharge a rechargeable power source of IMD 14. In the example of system 10, charging device 22 is the power transmitting unit and IMD 14 is the power receiving unit. IMD 14 may be in a flipped or non-flipped position.

Heat sink device 28 may include a housing that contains a phase change material. The housing may be configured to be removably attached to energy transfer coil 26. In this manner, the system may operate such that energy transfer coil 26 generates heat during a recharge session and the phase change material of heat sink device 28 absorbs at least a portion of the generated heat. When the phase change material is at the melting temperature, the heat may contribute to the heat of fusion of the phase change material and not to increasing the temperature of energy transfer coil 26.

A flexible coil of energy transfer coil 26 may be formed by one or more coils of wire. In one example the coil is formed by a wire wound into a spiral within a single plane (e.g., an in-plane spiral). This in-plane spiral may be constructed with a thickness equal to the thickness of the wire, and the in-plane spiral may be capable of transferring energy with another coil. In other examples, the coil may be formed by winding a coil into a spiral bent into a circle. However, this type of coil may not be as thin as the in-plane spiral. In some examples energy transfer coil 26 may include any one or more of the return-wrapping technique, a separation pad for e/h field protection in a passive recharge mattress pad, directional control of a multi-coil array and be controlled by an algorithm for recharge system coil activation, in any combination.

An issue that may occur is related to the electric field generated by the coil. Due to the voltage magnification across the coil when it resonates, the voltage developed across the coil is upwards of a thousand volts. This may factor into selection of proper insulation, but may also impact the emission of an excessive E-field as well. IEEE standard C95.1-2019 provides limits that govern the human exposure to electric, magnetic, and electromagnetic fields ranging from 0 Hz to 300 GHz. The electric field that the coil produces can be estimated by taking the voltage drop from the outermost turn to the innermost turn and dividing by the arclength that reaches out a distance to the patient. Previously, the E-field was exceeding the regulations by an order of 10³.

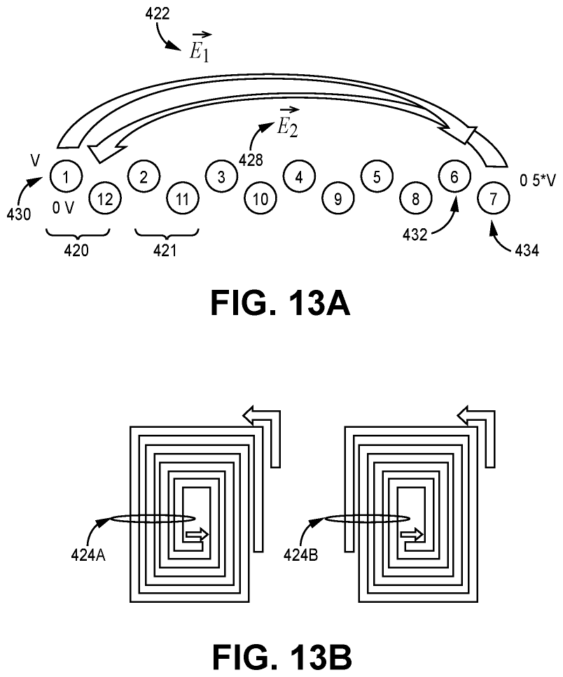

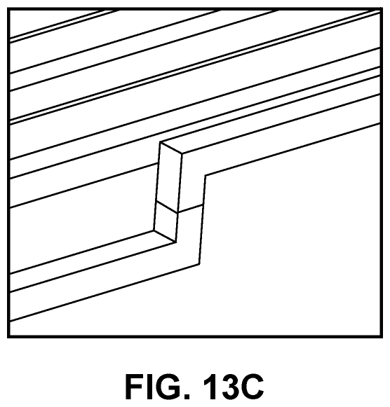

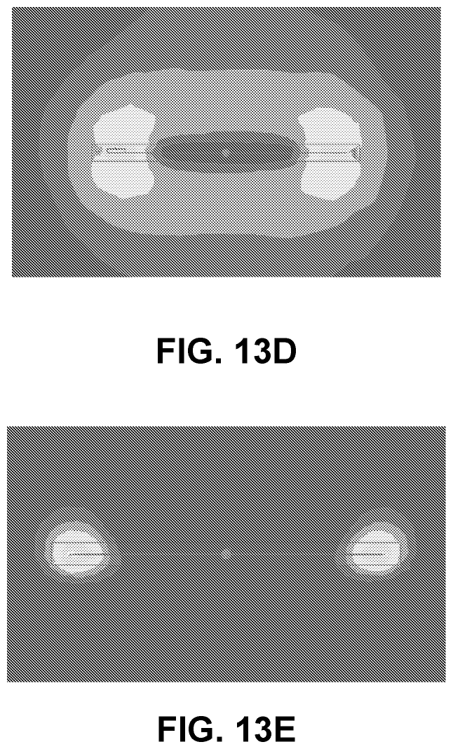

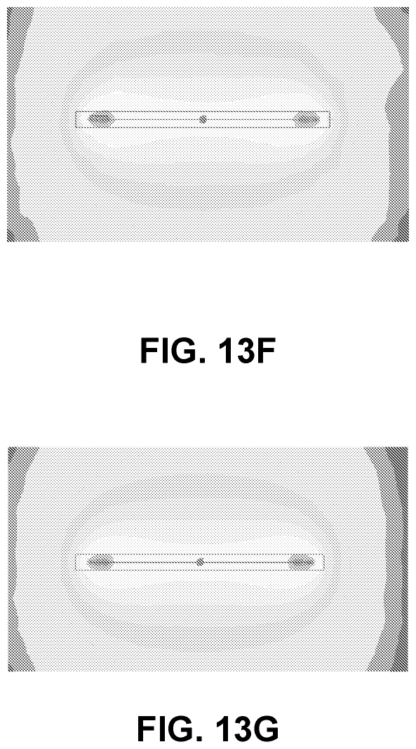

A different style of coil wrapping, e.g., return-wrapping, can help reduce the E-field exposure to the user. In some proposed designs, coils are simply wrapping the wire from the innermost turn to outermost. In contrast, in accordance with some examples described herein, energy transfer coil 26 includes a coil wrapped so that the turns wrap inwards and then back out again. The voltage drop across the coil would now be distributed half-and-half on the inwardly wrapped portion and the outwardly wrapped portion. In this way, two half-strength electric fields would still be generated, but the direction would be opposite and would cancel out via superposition. This wrapping method may nearly cancel out the near-field electric field generated while not affecting the magnetic field or effectiveness of the recharging process. As may be seen in FIGS. 13A and 13B, in some examples, the return wrapped coil strand may be located adjacent to its corresponding forward wrapped coil strand, e.g., coil 6 and coil 7 may be adjacent, coils 3 and 10 may be adjacent, and so on. Actual strand numbers may vary based on the coil size, number of wraps and other factors.

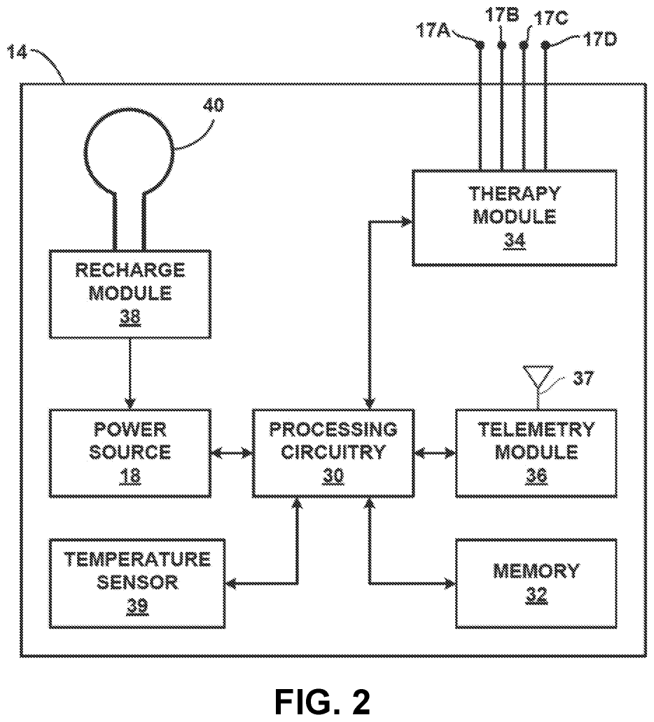

FIG. 2 is a block diagram illustrating example components of IMD 14 of FIG. 1. In the example illustrated in FIG. 2, IMD 14 includes temperature sensor 39, coil 40, processing circuitry 30, therapy module 34, recharge module 38, memory 32, telemetry module 36, and rechargeable power source 18. In other examples, IMD 14 may include a greater or a fewer number of components. In general, IMD 14 may comprise any suitable arrangement of hardware, alone or in combination with software and/or firmware, to perform the various techniques described herein attributed to IMD 14 and processing circuitry 30, and any equivalents thereof.

Processing circuitry 30 of IMD 14 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 14 may include a memory 32, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy module 34, recharge module 38, telemetry module 36, and temperature sensor 39 are described as separate modules, in some examples, some combination of processing circuitry 30, therapy module 34, recharge module 38, telemetry module 36 and temperature sensor 39 are functionally integrated. In some examples, processing circuitry 30, therapy module 34, recharge module 38, telemetry module 36, and temperature sensor 39 correspond to individual hardware units, such as ASICs, DSPs, FPGAs, or other hardware units.

Memory 32 may store therapy programs or other instructions that specify therapy parameter values for the therapy provided by therapy module 34 and IMD 14. In some examples, memory 32 may also store temperature data from temperature sensor 39, instructions for recharging rechargeable power source 18, thresholds, instructions for communication between IMD 14 and external charging device 22, or any other instructions required to perform tasks attributed to IMD 14. Memory 32 may be configured to store instructions for communication with and/or controlling one or more temperature sensors of temperature sensor 39. In various examples, memory 32 stores information related to determining the temperature of the housing and/or exterior surface(s) of the housing of IMD 14 based on temperatures sensed by one or more temperature sensors, such as temperature sensor 39, located within IMD 14.

For example, memory 32 may store one or more formulas, as further described below, that may be used to determine the temperature of the housing and/or exterior surface(s) of the housing based on temperature(s) sensed by the temperature sensor 39. Memory 32 may store values for one or more determined constants used by these formulas. Memory 32 may store instructions that, when executed by processing circuitry such as processing circuitry 30, perform an algorithm, including using the formulas, to determine a current temperature, or temperatures over time, for the housing and/or exterior surface(s) of the housing of IMD 14 during a charging session and/or for some time after a charging session performed on IMD 14. In some examples, memory 32 may store instructions that, when executed by processing circuitry such as processing circuitry 30, perform an algorithm, including using one or more formulas, to determine a value to be assigned to one or more of the constants used in the algorithm to determine a temperature for the housing and/or exterior surface(s) of the housing of IMD 14 during a charging session and/or for some time after a charging session performed on IMD 14.

Generally, therapy module 34 may generate and deliver electrical stimulation under the control of processing circuitry 30. In some examples, processing circuitry 30 controls therapy module 34 by accessing memory 32 to selectively access and load at least one of the stimulation programs to therapy module 34. For example, in operation, processing circuitry 30 may access memory 32 to load one of the stimulation programs to therapy module 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy module 34 uses to deliver the electrical stimulation signal. Therapy module 34 may be configured to generate and deliver electrical stimulation therapy via one or more of electrodes 17A. 17B, 17C, and 17D of lead 19. Alternatively, or additionally, therapy module 34 may be configured to provide different therapy to patient 12. For example, therapy module 34 may be configured to deliver drug delivery therapy via a catheter. These and other therapies may be provided by IMD 14.

IMD 14 also includes components to receive power from external charging device 22 to recharge rechargeable power source 18 when rechargeable power source 18 has been at least partially depleted. As shown in FIG. 2, IMD 14 includes secondary coil 40 and recharge module 38 coupled to rechargeable power source 18. Recharge module 38 may be configured to charge rechargeable power source 18 with the selected power level determined by either processing circuitry 30 or external charging device 22. Recharge module 38 may include any of a variety of charging and/or control circuitry configured to process or convert current induced in coil 40 into charging current to charge power source 18. Although processing circuitry 30 may provide some commands to recharge module 38, in some examples, processing circuitry 30 may not need to control any aspect of recharging.

Secondary coil 40 may include a coil of wire or other device capable of inductive coupling with a primary coil disposed external to patient 12. Although secondary coil 40 is illustrated as a simple loop of in FIG. 2, secondary coil 40 may include multiple turns of conductive wire. Secondary coil 40 may include a winding of wire configured such that an electrical current can be induced within secondary coil 40 from a magnetic field. The induced electrical current may then be used to recharge rechargeable power source 18. In this manner, the electrical current may be induced in secondary coil 40 associated with rechargeable power source 18. The induction may be caused by electrical current generated in the primary coil of external charging device 22, where the level of the current may be based on the selected power level. The coupling between secondary coil 40 and the primary coil of external charging device 22 may be dependent upon the alignment of the two coils. Generally, the coupling efficiency increases when the two coils share a common axis and are in close proximity to each other. External charging device 22 and/or IMD 14 may provide one or more audible tones or visual indications of the alignment.

Although inductive coupling is generally described as the method for recharging rechargeable power source 18, other wireless energy transfer techniques may alternatively be used. Any of these techniques may generate heat in IMD 14 such that the charging process may need to be controlled by matching the determined temperature to one or more thresholds, modeling tissue temperatures based on the determined temperature, or using a calculated cumulative thermal dose as feedback.

Recharge module 38 may include one or more circuits that process, filter, convert and/or transform the electrical signal induced in the secondary coil to an electrical signal capable of recharging rechargeable power source 18. For example, in alternating current induction, recharge module 38 may include a half-wave rectifier circuit and/or a full-wave rectifier circuit configured to convert alternating current from the induction to a direct current for rechargeable power source 18. The full-wave rectifier circuit may be more efficient at converting the induced energy for rechargeable power source 18. However, a half-wave rectifier circuit may be used to store energy in rechargeable power source 18 at a slower rate. In some examples, recharge module 38 may include both a full-wave rectifier circuit and a half-wave rectifier circuit such that recharge module 38 may switch between each circuit to control the charging rate of rechargeable power source 18 and temperature of IMD 14.

Rechargeable power source 18 may include one or more capacitors, batteries, and/or other energy storage devices. Rechargeable power source 18 may deliver operating power to the components of IMD 14. In some examples, rechargeable power source 18 may include a power generation circuit to produce the operating power. Rechargeable power source 18 may be configured to operate through many discharge and recharge cycles. Rechargeable power source 18 may also be configured to provide operational power to IMD 14 during the recharge process. In some examples, rechargeable power source 18 may be constructed with materials to reduce the amount of heat generated during charging. In other examples, IMD 14 may be constructed of materials and/or using structures that may help dissipate generated heat at rechargeable power source 18, recharge module 38, and/or secondary coil 40 over a larger surface area of the housing of IMD 14.

Although rechargeable power source 18, recharge module 38, and secondary coil 40 are shown as contained within the housing of IMD 14, in alternative implementations, at least one of these components may be disposed outside of the housing. For example, in some implementations, secondary coil 40 may be disposed outside of the housing of IMD 14 to facilitate better coupling between secondary coil 40 and the primary coil of external charging device 22. These different configurations of IMD 14 components may allow IMD 14 to be implanted in different anatomical spaces or facilitate better inductive coupling alignment between the primary and secondary coils.

IMD 14 may also include temperature sensor 39. Temperature sensor 39 may include one or more temperature sensors configured to measure the temperature of respective portions of IMD 14. As described herein, these temperature sensor(s) may not be thermally coupled to, and may not be directly attached to, the portion of the device for which a temperature is to be determined based on the sensed temperature measured by temperature sensor 39. In one instance, the temperature sensor is not directly attached to the housing or to the exterior surface(s) of the housing of the device. In other words, temperature measurement is not performed through direct contact or physical contact between the temperature sensor and the target portion to be measured. Although the temperature sensor may be physically attached to the target portion or target surface through one or more structures, thermal conduction that may occur between the target portion and the sensor is not directly used to measure the temperature of the target portion.

Temperature sensor 39 may be arranged to measure the temperature of a component, surface, or structure, e.g., secondary coil 40, power source 18, recharge module 38, and other circuitry housed within IMD 14. Temperature sensor 39 may be disposed internal of the housing of IMD 14 or otherwise disposed relative to the external portion of housing (e.g., tethered to an external surface of housing via an appendage cord, light pipe, heat pipe, or some other structure). As described herein, temperature sensor 39 may be used to make temperature measurements of internal portions of the IMD 14, the temperature measurements used as a basis for determining the temperature of the housing and/or external surface of IMD 14. For example, processing circuitry 30 or processing circuitry of external charging device 22 may use these temperature measurements to determine the housing/external surface temperatures of IMD 14. In other examples, temperature measurements may be used to determine temperatures of a specific portion of the housing or a component coupled thereto, such as header block 15, or another module that is coupled to IMD 14. For instance, IMD 14 may comprise an additional housing that is separate from, but affixed to, the housing that contains some components of IMD 14. As one specific example, a secondary coil such as secondary coil 40 may reside within an additional housing that is external to, but affixed to, main the housing. Temperature measurements may be used to determine a temperature of a surface or portion of this additional housing or a structure within this housing such as the secondary coil itself. As another example, IMD 14 may carry an appendage protruding from the housing carrying one or more electrodes that serves as a stub lead for delivering electrical stimulation therapy. Temperature sensor 39 may be used to make temperature measurements that may be used as a basis for determining the temperature of a portion of this structure. The determined temperatures are then further used as feedback to control the power levels or charge times (e.g., cycle times) used during the charging session of rechargeable power source 18. In some examples, temperature sensor 39 may be used to obtain temperature measurements of a header block 15, or another module that is coupled to IMD 14. For instance, IMD 14 may comprise an additional housing that is separate from, but affixed to, the housing that contains some components of IMD 14. As one specific example, a secondary coil may reside within an additional housing. As another example, IMD 14 may carry an appendage protruding from the housing carrying one or more electrodes that serves as a stub lead for delivering electrical stimulation therapy. Temperature sensor 39 may be used to make temperature measurements that may be used as a basis for determining the temperature of a surface, or another portion, of these and other structures.

Although a single temperature sensor may be adequate, multiple temperature sensors may provide more specific temperature readings of separate components or of different portions of the IMD. Although processing circuitry 30 may continuously measure temperature using temperature sensor 39, processing circuitry 30 may conserve energy by only measuring temperatures during recharge sessions. Further, temperatures may be sampled at a rate necessary to effectively control the charging session, but the sampling rate may be reduced to conserve power as appropriate. Processing circuitry 30 may be configured to access memory, such as memory 32, to retrieve information comprising instructions, formulas, determined values, and/or one or more constants, and to use this information to execute an algorithm to determine a current temperature, and/or a series of temperatures over time, for the housing and/or exterior surface(s) of the housing of IMD 14 based on the measured temperature(s) provided by temperature sensor 39.

Processing circuitry 30 may also control the exchange of information with external charging device 22 and/or an external programmer using telemetry module 36. Telemetry module 36 may be circuitry configured for wireless communication using radio frequency protocols, such as BLUETOOTH, or similar RF protocols, as well as using inductive communication protocols. Telemetry module 36 may include one or more antennas 37 configured to communicate with external charging device 22, for example. Processing circuitry 30 may transmit operational information and receive therapy programs or therapy parameter adjustments via telemetry module 36. Also, in some examples, IMD 14 may communicate with other implanted devices, such as stimulators, control devices, or sensors, via telemetry module 36. In addition, telemetry module 36 may be configured to control the exchange of information related to sensed and/or determined temperature data, for example temperatures sensed by and/or determined from temperatures sensed using temperature sensor 39. In some examples, telemetry module 36 may communicate using inductive communication, and in other examples, telemetry module 36 may communicate using RF frequencies separate from the frequencies used for inductive charging.

In some examples, processing circuitry 30 may transmit additional information to external charging device 22 related to the operation of rechargeable power source 18. For example, processing circuitry 30 may use telemetry module 36 to transmit indications that rechargeable power source 18 is completely charged, rechargeable power source 18 is fully discharged, or any other charge status of rechargeable power source 18. In some examples, processing circuitry 30 may use telemetry module 36 to transmit instructions to external charging device 22, including instructions regarding further control of the charging session, for example instructions to lower the power level or to terminate the charging session, based on the determined temperature of the housing/external surface 19 of the IMD.

Processing circuitry 30 may also transmit information to external charging device 22 that indicates any problems or errors with rechargeable power source 18 that may prevent rechargeable power source 18 from providing operational power to the components of IMD 14. In various examples, processing circuitry 30 may receive, through telemetry module 36, instructions for algorithms, including formulas and/or values for constants to be used in the formulas, that may be used to determine the temperature of the housing and/or exterior surface(s) of the housing of IMD 14 based on temperatures sensed by temperature sensor 39 located within IMD 14 during and after a recharging session performed on rechargeable power source 18.

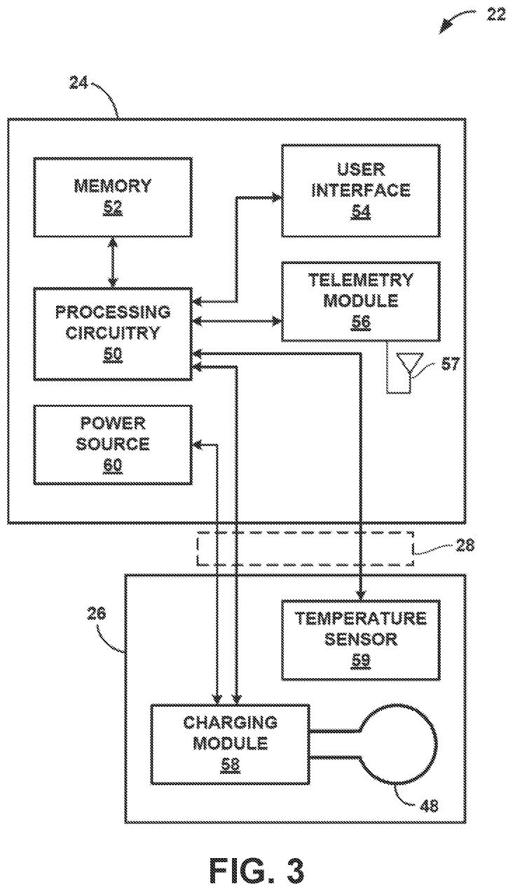

FIG. 3 is a block diagram of an example external charging device 22 of FIG. 1. While external charging device 22 may generally be described as a hand-held device, external charging device 22 may be a larger portable device or a more stationary device. In addition, in other examples external charging device 22 may be included as part of an external programmer or include functionality of an external programmer. External charging device 22 may also be configured to communicate with an external programmer. As shown in FIG. 3, external charging device 22 includes two separate components. Housing 24 encloses components such as a processing circuitry 50, memory 52, user interface 54, telemetry module 56, and power source 60. Charging head 26 may include charging module 58, temperature sensor 59, and coil 48. As shown in FIG. 2, housing 24 is electrically coupled to charging head 26 via charging cable 27.

A separate charging head 26 may facilitate optimal positioning of coil 48 over coil 40 of IMD 14. However, charging module 58 and/or coil 48 may be integrated within housing 24 in other examples. Memory 52 may store instructions that, when executed by processing circuitry 50, causes processing circuitry 50 and external charging device 22 to provide the functionality ascribed to external charging device 22 throughout this disclosure, and/or any equivalents thereof.

External charging device 22 may also include one or more temperature sensors, illustrated as temperature sensor 59, similar to temperature sensor 39 of FIG. 2. As shown in FIG. 3, temperature sensor 59 may be disposed within charging head 26. In other examples, one or more temperature sensors of temperature sensor 59 may be disposed within housing 24. For example, charging head 26 may include one or more temperature sensors positioned and configured to sense the temperature of coil 48 and/or a surface of the housing of charging head 26. In some examples, external charging device 22 may not include temperature sensor 59.

In general, external charging device 22 comprises any suitable arrangement of hardware, alone or in combination with software and/or firmware, to perform the techniques ascribed to external charging device 22, and processing circuitry 50, user interface 54, telemetry module 56, and charging module 58 of external charging device 22, and/or any equivalents thereof. In various examples, external charging device 22 may include one or more processors, such as one or more microprocessors, DSPs, ASICs, FPGAs. or any other equivalent integrated or discrete logic circuitry, as well as any combinations of such components. External charging device 22 also, in various examples, may include a memory 52, such as RAM, ROM, PROM, EPROM, EEPROM, flash memory, a hard disk, a CD-ROM, comprising executable instructions for causing the one or more processors to perform the actions attributed to them. Moreover, although processing circuitry 50, telemetry module 56, charging module 58, and temperature sensor 59 are described as separate modules, in some examples, processing circuitry 50, telemetry module 56, charging module 58, and/or temperature sensor 59 are functionally integrated. In some examples, processing circuitry 50, telemetry module 56, charging module 58, and/or temperature sensor 59 correspond to individual hardware units, such as ASICs, DSPs, FPGAs, or other hardware units.

Memory 52 may store instructions that, when executed by processing circuitry 50, cause processing circuitry 50 and external charging device 22 to provide the functionality ascribed to external charging device 22 throughout this disclosure, and/or any equivalents thereof. For example, memory 52 may include instructions that cause processing circuitry 50 to control the power level used to charge IMD 14 in response to the determined temperatures for the housing/external surface(s) of IMD 14, as communicated from IMD 14, or instructions for any other functionality. In addition, memory 52 may include a record of selected power levels, sensed temperatures, determined temperatures, or any other data related to charging rechargeable power source 18. Processing circuitry 50 may, when requested, transmit any of this stored data in memory 52 to another computing device for review or further processing. Processing circuitry 50 may be configured to access memory, such as memory 32 of IMD 14 and/or memory 52 of external charging device 22, to retrieve information comprising instructions, formulas, and determined values for one or more constants, and to use this information to perform an algorithm to determine a current temperature, and/or a series of temperatures over time, for the housing and/or exterior surface(s) of the housing of IMD 14 based on the measured temperature(s) provided by temperature sensors 39 of IMD 14.

Memory 52 may be configured to store instructions for communication with and/or control of one or more temperature sensors 39 of IMD 14. In various examples, memory 52 stores information related to determining the temperature of the housing and/or exterior surface(s) of the housing of IMD 14 based on temperatures sensed by one or more temperature sensors, such as temperature sensors 39, located within IMD 14. For example, memory 52 may store one or more formulas, as further described below, that may be used to determine the temperature of the housing and/or exterior surface(s) of the housing based on temperature(s) sensed by the temperature sensors 39. Memory 52 may store values for one or more determined constants used by these formulas. Memory 52 may store instructions that, when executed by processing circuitry such as processing circuitry 50, performs an algorithm, including using the formulas, to determine a current temperature, or a series of temperatures over time, for the housing and/or exterior surface(s) of the housing of IMD 14 during a charging session and/or for some time after a charging session performed on IMD 14. In some examples, memory 52 may store instructions that, when executed by processing circuitry such as processing circuitry 50, perform an algorithm, including using one or more formulas, to determine a value to be assigned to one or more of the constants used in the algorithm used to determine the temperature(s) associated with the housing and/or exterior surface(s) of the housing of IMD 14 during a charging session and/or for some time after a charging session performed on IMD 14.

User interface 54 may include a button or keypad, lights, a speaker for voice commands, a display, such as a liquid crystal (LCD), light-emitting diode (LED), or cathode ray tube (CRT). In some examples, the display may be a touch screen. As discussed in this disclosure, processing circuitry 50 may present and receive information relating to the charging of rechargeable power source 18 via user interface 54. For example, user interface 54 may indicate when charging is occurring, quality of the alignment between coils 40 and 48, the selected power level, current charge level of rechargeable power source 18, duration of the current recharge session, anticipated remaining time of the charging session, sensed temperatures, or any other information. Processing circuitry 50 may receive some of the information displayed on user interface 54 from IMD 14 in some examples. In some examples, user interface 54 may provide an indication to the user regarding the quality of alignment between coils 40, depicted in FIG. 2 and coil 48, based on the charge current to the battery.

User interface 54 may also receive user input via user interface 54. The input may be, for example, in the form of pressing a button on a keypad or selecting an icon from a touch screen. The input may request starting or stopping a recharge session, a desired level of charging, or one or more statistics related to charging rechargeable power source 18 (e.g., the cumulative thermal dose). User input may also include inputs related to temperature thresholds for the IMD that may be used to regulate for example a maximum housing/surface temperature the patient is willing to experience during a charging session of the IMD. The inputs related to threshold values may be store in memory 52, and/or transmitted through telemetry module 56 to IMD 14 for storage in a memory, such as memory 32, located within IMD 14. In this manner, user interface 54 may allow the user to view information related to the charging of rechargeable power source 18 and/or receive charging commands, and to provide inputs related to the charging process.

External charging device 22 also includes components to transmit power to recharge rechargeable power source 18 associated with IMD 14. As shown in FIG. 3, external charging device 22 includes primary coil 48 and charging module 58 coupled to power source 60. Charging module 58 may be configured to generate an electrical current in primary coil 48 from electrical energy stored in or provided by power source 60. Although primary coil 48 is illustrated as a simple loop in FIG. 3, primary coil 48 may include multiple turns of wire. Charging module 58 may generate the electrical current according to a power level selected by processing circuitry 50 based on the sensed and/or determined temperature or temperatures received from IMD 14 and/or a temperature sensor within external charging device 22. As described herein, processing circuitry 50 may select a “high” power level, a “low” power level, or a variety of different power levels to control the rate of recharge in rechargeable power source 18 and the temperature of IMD 14. In some examples, processing circuitry 50 may control charging module 58 based on a power level selected by processing circuitry 30 of IMD 14. The determined temperature of the housing and/or exterior surface(s) of the housing of IMD 14 used as feedback for control of the recharge power level may be derived from a temperature sensed by a temperature sensor within IMD 14. Although processing circuitry 50 may control the power level used for charging rechargeable power source 18, charging module 58 may include processing circuitry including one or more processors configured to partially or fully control the power level based on the determined temperatures.

Primary coil 48 may include a coil of wire, e.g., having multiple turns, or other devices capable of inductive coupling with a secondary coil 40 disposed within patient 12. Primary coil 48 may include a winding of wire configured such that an electrical current generated within primary coil 48 can produce a magnetic field configured to induce an electrical current within secondary coil 40. The induced electrical current may then be used to recharge rechargeable power source 18. In this manner, the electrical current may be induced in secondary coil 40 associated with rechargeable power source 18. The coupling efficiency between secondary coil 40 and primary coil 48 of external charging device 22 may be dependent upon the alignment of the two coils. Generally, the coupling efficiency increases when the two coils share a common axis and are in close proximity to each other. User interface 54 of external charging device 22 may provide one or more audible tones or visual indications of the alignment.

Charging module 58 may include one or more circuits that generate an electrical signal, and an electrical current, within primary coil 48. Charging module 58 may generate an alternating current of specified amplitude and frequency in some examples. In other examples, charging module 58 may generate a direct current. In any case, charging module 58 may be capable of generating electrical signals, and subsequent magnetic fields, to transmit various levels of power to IMD 14. In this manner, charging module 58 may be configured to charge rechargeable power source 18 of IMD 14 with the selected power level.

The power level that charging module 58 selects for charging may be used to vary one or more parameters of the electrical signal generated for coil 48. For example, the selected power level may specify wattage, electrical current of primary coil 48 or secondary coil 40, current amplitude, voltage amplitude, pulse rate, pulse width, a cycling rate, or a duty cycle that determines when the primary coil is driven, or any other parameter that may be used to modulate the power transmitted from coil 48. In this manner, each power level may include a specific parameter set that specifies the signal for each power level. Changing from one power level to another power level (e.g., a “high” power level to a lower power level) may include adjusting one or more parameters. For instance, at a “high” power level, the primary coil may be substantially continuously driven, whereas at a lower power level, the primary coil may be intermittently driven such that periodically the coil is not driven for a predetermined time to control heat generation. The parameters of each power level may be selected based on hardware characteristics of external charging device 22 and/or IMD 14.

Power source 60 may deliver operating power to the components of external charging device 22. Power source 60 may also deliver the operating power to drive primary coil 48 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

External charging device 22 may include one or more temperature sensors shown as temperature sensor 59 (e.g., similar to temperature sensor 39 of IMD 14) for sensing the temperature of a portion of the device. For example, temperature sensor 59 may be disposed within charging head 26 and oriented to sense the temperature of the housing of charging head 26. In another example, temperature sensor 59 may be disposed within charging head 26 and oriented to sense the temperature of charging module 58 and/or coil 48. In other examples, external charging device 22 may include multiple temperature sensors 59 each oriented to any of these portions of device to manage the temperature of the device during charging sessions.

Telemetry module 56 supports wireless communication between IMD 14 and external charging device 22 under the control of processing circuitry 50. Telemetry module 56 may also be configured to communicate with another computing device via wireless communication techniques, or direct communication through a wired connection. In some examples, telemetry module 56 may be substantially similar to telemetry module 36 of IMD 14 described herein, providing wireless communication via an RF or proximal inductive medium. In some examples, telemetry module 56 may include an antenna 57, which may take on a variety of forms, such as an internal or external antenna. Although telemetry modules 56 and 36 may each include dedicated antennas for communications between these devices, telemetry modules 56 and 36 may instead, or additionally, be configured to utilize inductive coupling from coils 40 and 48 to transfer data.

Examples of local wireless communication techniques that may be employed to facilitate communication between external charging device 22 and IMD 14 include radio frequency and/or inductive communication according to any of a variety of standard or proprietary telemetry protocols, or according to other telemetry protocols such as the IEEE 802.11x or Bluetooth specification sets. In this manner, other external devices may be capable of communicating with external charging device 22 without needing to establish a secure wireless connection. As described herein, telemetry module 56 may be configured to receive a signal or data representative of a sensed temperature from IMD 14 or a determined temperature of the housing and/or exterior surface(s) of the housing of the IMD based on the sensed temperature. The determined temperature may be determined using an algorithm, including use of formula(s) as further described below, based on measuring the temperature of the internal portion(s) of the IMD, such as circuitry mounted to a circuit board located within IMD 14. In some examples, multiple temperature readings by IMD 14 may be averaged or otherwise used to produce a single temperature value that is transmitted to external charging device 22. The sensed and/or determined temperature may be sampled and/or transmitted by IMD 14 (and received by external charging device 22) at different rates, e.g., on the order of microseconds, milliseconds, seconds, minutes, or even hours. Processing circuitry 50 may then use the received temperature information to control charging of rechargeable power source 18 (e.g., control the charging level used to recharge power source 18).

In one or more examples, the functions described in this disclosure may be implemented in hardware, software, firmware, or any combination thereof. For example, the various components of FIGS. 2 and 3, such as processing circuitry 30 and processing circuitry 50 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache).

By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor,” as used herein, such as ECS controller 202, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

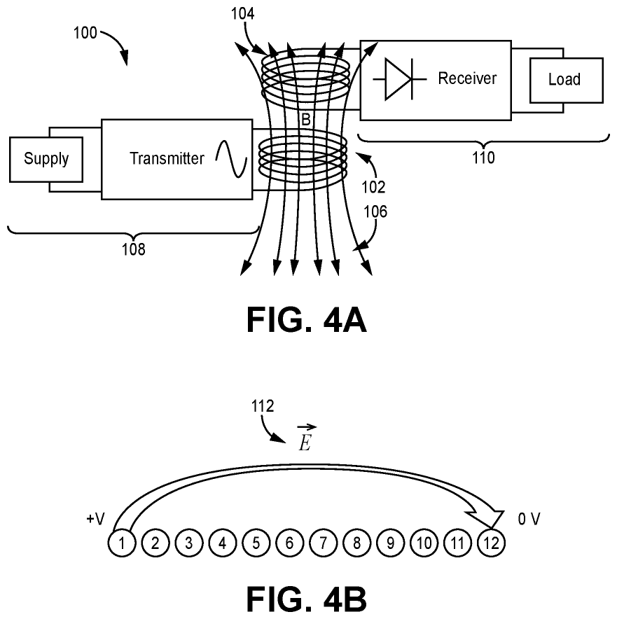

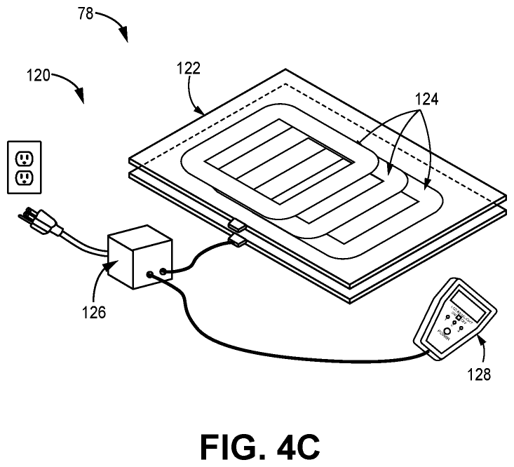

FIGS. 4A and 4B are conceptual diagrams illustrating primary and secondary coils of a wireless power transfer system along with associated magnetic field and electric field. System 100 is an example of system 10 described above in relation to FIG. 1. Power transmitting device 108 with primary coil 102 and power receiving device 110 with secondary coil 104 correspond to charging device 22 with energy transfer coil 26 and IMD 14 with secondary coil 16, respectively. Power transmitting device 108 provides AC current to primary coil 102 to generate magnetic field 106. Magnetic field 106 generates and electric current in secondary coil 104. Power receiving device 110 may use the received wireless power to charge an electrical energy storage device, such as power source 18 described above in relation to FIG. 2.

The SI unit for magnetic flux is the weber. Two different vectors may represent a magnetic field. Magnetic flux density, or magnetic induction, is symbolized by B, while magnetic field strength, or magnetic field intensity, is symbolized by H. In this disclosure, either B or H may describe the magnetic field used in wireless power system 100.

The AC current supplied to primary coil 102 also generates an electric field 112, as shown by FIG. 4B. As described herein, one or more techniques of this disclosure may be directed to providing a magnetic field over a large area while keeping the electric field 112 within safe levels.

In some examples, the recharging process may last as long as an hour everyday which is time consuming and potentially uncomfortable for the user. In the example of rechargeable and implantable medical devices, a wireless power transfer system may include features for charging the device, e.g., IMD 14 of FIGS. 1 and 2, and limiting the electric and magnetic field dosages on a user during a charging cycle. Other features may include adhering to thermal safety standards be ensuring the IMD and antenna temperature, e.g., primary coil 102 and secondary coil 104, does not exceed safe temperature levels, such as 41° C. for longer than 21 minutes.

FIG. 4C is a conceptual diagram illustrating an example multi-coil recharging pad. System 120 is an example of wireless power transmitting device 102 and primary coil 102 of FIG. 4A and external charging device 22 described above in relation to FIGS. 1 and 3. In the example of FIG. 4C, system 120 includes a pad 122 with one or more primary coils 124, circuitry 126 and remote control 128. The circuitry described above in relation to FIG. 3 may be located in the box indicated by 126 as well as in pad 122, and remote control 128.

A charging mattress pad, such as system 120, may be incorporated into an individual's sleep cycle and relatively easily into a user's everyday life. System 120, which may also be referred to as device 120 or charging device 120, is hands-free, which is not the case for some other types charger systems. The example mattress pad 122 of FIG. 4C allows for multiple large square coils 124 to provide sufficient power to the device a range of distances between coils 124 and the secondary coil of the power receiving device. The large coils transfer energy through the magnetic fields created with AC current from a standard outlet. With the three large coils, the range of total effective charging area may include exceed five square feet (ft) at approximately 10 cm distance and two ft² at a 20 cm distance, which may enable more mobility for the user. In some examples, processing circuitry of system 120 may execute programming instructions to automatically select the most effective of three coils implanted in the mattress pad using a variety of sensors and control algorithms designed using mathematical models and computer simulations. Other sizes and dimensions for primary coils 124 may be desirable for other charging systems, such as a pad placed on a chair, in the seat of a car and so on.

Large coil designs emit larges dosages of electric and magnetic fields which may prove to be harmful for the user in some rare instances. In some examples coils 124 of system 120 may incorporates a return wrap style design that cancels portions of the electric field while providing a relatively strong magnetic field to ensure charging, thus meeting the electric and magnetic requirement and safety standard.

Neurostimulators are often used by individuals that have chronic pain, Parkinson's disease, muscle dystrophy, or other neurological conditions. The neurostimulator can be placed within the user's trunk cavity in the pectoral, abdominal, or lumbar region. Depending on the electrical stimulation needed by the user, the neurostimulator implant may be used often and require charging. Power can be transferred wirelessly through the body, e.g., transcutaneous charging, using oscillating magnetic fields. For example, a user may hold a charging device 1 to 3 centimeters from the implant location for approximately one hour. With some neurostimulators, the effective charging area of is approximately 6.5 cm² which is comparable the size of a U.S. quarter. Because of the physical states of the neurostimulator users, the charging operation can be time consuming.

The devices, systems, and techniques of the present disclosure may provide advantages over other types of systems and help minimize the burden of charging an implantable medical device and enable charging to be easily integrated into the life activities of a patient. Additionally, the devices, systems, and techniques may help increase the target charging distance, e.g., from 3 cm to 10 to 20 cm, as well as making the product more user friendly by having the recharge process be less intrusive to the patient's daily routine.

In some charging devices and systems, induction charging may wirelessly transfer power to the implanted medical device, or other types of power receiving unit. As described above in relation to FIG. 4A, a transmitting coil is induced with AC current and creates a magnetic flux that produces an AC current in the receiving coil that can be converted to DC current using a rectifier. The produced DC current can be used to charge the battery of the implant, e.g., IMD 14 of FIGS. 1 and 2. In some examples of charging systems, the transmitting coil may function best when located within three centimeters (cm) of the receiving coil and centered and rotated in orientation to efficiently charge the implant. Even with an efficient charging position, the recharge process can take up to an hour a day. Some examples of IMDs and transmitting coils may not be interchangeably used from one company to another.

The present disclosure describes, in some examples, a wireless-power transfer (WPT) charging system for devices, such as implantable medical devices. The system may provide advantages over other types of WPT systems by reducing the charging time e.g., to 30 minutes or less, decreasing physical movement restrictions on patients during the charging process, integrating the charging system into the patient's everyday life, and any combination thereof.

Some example advantages achieved by the disclosed devices, systems, and techniques may include:

Example Technical Goals (any one or more of the following):
1. System 120 may operate using standard line power such 120 VAC 60 Hz, 230V 50 Hz, 100V 50 Hz and so on.
2. In some examples, system 120 may draw 15 amps or less from the line supply.
3. System 120 may conform to IEC 60601-1 for electrical safety and performance of medical equipment.
4. The power supply may offer internal overcurrent protection.
5. System 120 may not cause harmful interference as per FCC standards.
6. The antenna of system 120 e.g., primary coils 124, may operate at a frequency that resonates with the implanted device at the intended resonant frequency.
7. System 120 may operate at a frequency in so that it does not interfere with other electrical devices or other signals as per FCC standards.
8. Processing circuitry of system 120 may prevent the internal battery of the implant from becoming overcharged.
9. System 120 may not be affected by electrostatic discharge.

Example Safety Goals (any one or more of the following):

1. System 120 may limit the thermal dose on the muscle and fat to no more than 40 minutes of cumulative equivalent minutes at 43° C. per ISO 14708-3: 2017, clause 17.1.
2. System 120 may limit the thermal dose on the skin to no more than 21 minutes of cumulative equivalent minutes at 43° C. per ISO 14708-3:2017, clause 17.1.
3. System 120 may stop transmitting power when the implant reaches a temperature of 41° C.
4. The circuitry enclosure of system 120 are configured to prevent the user from electrical shock hazards.
5. System 120 may stop providing power when the presence of the implant is not detected for longer than a threshold duration, e.g., approximately five minutes.

Example Usability Goals (any one or more of the following):
1. The system should charge the medical device at a distance of 10 to 20 cm.
2. The system should have an effective charging area of 929 cm² or more.
3. The system should be intended only for the use of recharging an implantable device.
4. The system should charge through clothing less than or equal to 1 cm thick.

Comfort of User Goals (any one or more of the following):
1. System 120 may have a setup time of less than five (5) minutes before every charging cycle.
2. System 120 should charge from a distance in the range of approximately 5 cm to 25 cm away from the implantable device.
3. System 120 may have less than five (5) buttons for activation.
4. System 120 may charge the battery in less than 60 minutes if patient needs to be stationary, or five (5) hours if an effective charging area of 1 ft²-3 ft² is created.

Applicable Standards (any one or more of the following):
1. IEC 60601-1—Medical electrical equipment—Part 1: General requirements for basic safety and essential performance

2. ISO 14708-3:2017 clause 17.1—Implants for surgery—Active implantable medical devices—Part 3: Implantable neurostimulators

3. IEEE Std C95.1-2019—IEEE Standard for Safety Levels with Respect to Human Exposure to Electric, Magnetic, and Electromagnetic Fields, 0 Hz to 300 GHz

4. Federal Communications Commission (FCC) rules and regulations

5. Title 47 Part 15—Regulation on the operation of intentional, unintentional, or incidental radiators without an individual license.
6. Title 47 Part 18—Industrial, scientific, and Medical Equipment

In examples described herein, three primary coils 124 are used to charge the implantable neurostimulator device. In other examples, more coils or fewer coils may be included in system 120. The transmitting coils may specifically dimensioned to be efficient in inducing current in the receiving coil of the neurostimulator device based on the application of system 120, e.g., in a mattress, a chair or couch, a vehicle seat, and so on. In the example of a mattress, a mattress pad may separate the user at approximately 10 cm from the coils. Therefore, the charging distance may be between 20 cm and 30 cm considering that the mattress pad spaces in some examples. System 120 may be configured dynamically during a charging session to deliver wireless electrical energy to a power receiving device based on the implant's battery voltage, charge current, casing temperature, power transfer efficiency, calculated heating and other system metrics.

In some examples, a charging mattress pad is designed to keep the user a safe distance from the charging coils. Examples of system 120 may provide charging while the user sleeps. Incorporated into the mattress pad are multiple square copper coils that wirelessly transmit power to a neurostimulator device, or other implantable device, and a distance from the surface of the mattress. To achieve the range and effective charging area, in the example of system 120, three square transmitting coils are placed within the mattress pad with dimensions of 76 cm by 76 cm. Driving circuitry, controlled by processing circuitry, e.g., as described above in relation to FIGS. 1-3, may be configured switch between which of the three coils is connected and powered.

The user may begin a charging cycle by operating remote control 128, in the example of FIG. 4A. In some examples, a charging cycle for system 120 may have a communications stage, a probing stage, an active charging phase, and as needed a thermal cooldown stage. A user interface on remote control 128 may prompt the microcontroller to transition from its standby mode and establish communication with the user's neurostimulator device. Once communication is established, the implant's current battery voltage will be sent to the processing circuitry, e.g., a microcontroller of system 120. If the battery voltage for the power receiving device is less than a threshold. e.g., 2.5-2.8 V (volts) for some types of batteries, and is in need of charging, then the user interface on remote control 128 may prompt the user to lie down on pad 122 and begin to charge the INS device.

Upon entering the active charging stage, the system 120 will check that the neurostimulator device's casing temperature is currently under a threshold temperature, such as 39° C.-43° C., and that the battery voltage is still less than the “charging needed” threshold, e.g., a battery voltage of under 2.8 V. When both temperature and battery voltage conditions are met, then system 120 may enter probing stage, where it puts a current between one and amps peak AC. e.g., approximately two amps, through the first coil of coils 124 for a specified duration of about one to 20 seconds. The primary coils 124 may also be referred to as transmit coils or Tx coils in this disclosure.

Processing circuitry of system 120, e.g., processing circuitry 50 described above in relation to FIG. 3, may receive via telemetry module 56 an average charging current induced to the battery during that period, e.g., power source 18 via the circuitry of recharge module 38. The processing circuitry may then shut down the current to the first primary coil and repeat the probing process for the second and third Tx coils. In some examples, the processing circuitry may compare the average charging currents that each Tx coil induces. The processing circuitry may activate whichever Tx coil induces the greatest average charging current for a predetermined active charging duration, e.g., about five to twenty minutes. In some examples the probing stage run for a duration of a few seconds to a minute or so. In some examples, after the active charging stage, the processing circuitry may repeat the probing stage and return to the active charging stage. Repeating the probing stage and active charging stage may account for any movement of the patient during the charging session. In some examples, the duration of each stage, the threshold levels and so on may be adjustable by the user, e.g., via remote control 128, or some other user interface of system 100, as described above in relation to FIG. 1.

If at any time the calculated heat of the implant and/or a measured temperature exceeds a threshold, system 120 may enters a thermal cooldown stage in which all of coils 124 may be deactivated for a specified duration, e.g., five minutes, ten minutes, or longer, to allow the implant to cool down. The length of the period for the thermal cooldown stage may also be adjusted based on performance testing. The thermal cooldown stage may also be referred to as the thermal shutdown stage in this disclosure.

In some examples, if at any time the communication with the implant is lost, the system may deactivate all Tx coils, prompts the user via a user interface, e.g., an audio alarm, via remote control 128 and so on, and may enter the standby mode. The system may also enter standby mode upon fully charging the battery above a threshold charge level, e.g., a measured battery voltage. When system 120 determines the battery is fully charged, system 120 may also prompt the user that the implant was fully charged via a user interface using visual, audio, vibration or other notification. In some examples, the implant may adjust the electrical stimulation to provide a haptic sensation (e.g., a perceptible stimulus that may feel different than other perceivable therapeutic stimulation) to alert the user, e.g., for communication loss, end of charging, and so on.

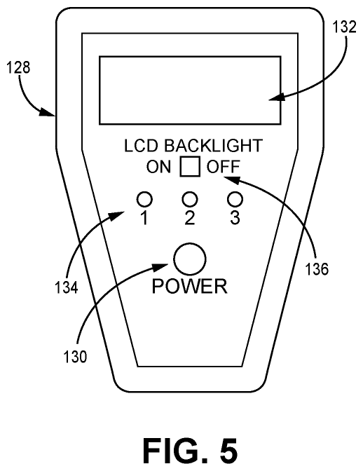

FIG. 5 is a conceptual diagram illustrating an example remote control for example wireless power transfer systems of this disclosure. Remote control 128 of FIG. 5 is an example implementation of remote control 128 described above in relation to FIG. 4C. A patient may use remote control 128 to control and monitor the charging process.

In the example of FIG. 5, remote control 128 includes a user interface with power button 130, three LED indicator lights 134, which may be colored for example green, blue, and red to indicate the state of charging as well as warnings and errors to the user. In some examples indicator lights 134 may also be labeled with numbers (e.g., 1, 2, and 3) for users that may be color blind, or otherwise cannot identify a color. The user interface may also include display screen 132 that provides written prompts to the user, and a switch 136 that operates the backlight of display screen 132.

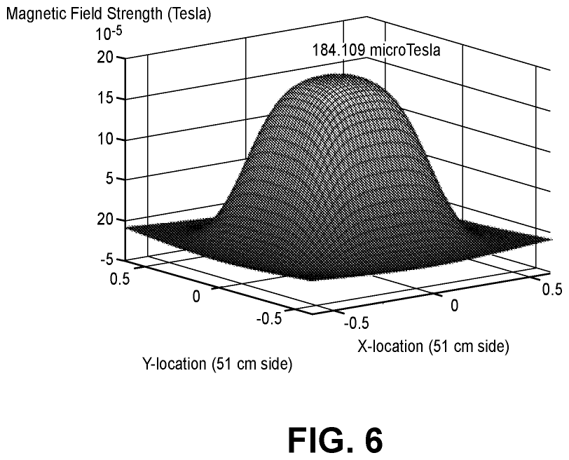

FIG. 6 is a conceptual diagram illustrating an example magnetic field strength of a single coil. The example of FIG. 6 illustrates a simulation of an example square-shaped coil similar to primary coils 124 described above in relation to FIG. 4C, as well as the primary coils described above in relation to FIGS. 1, 3, 4A and 4B. The example implementation of FIG. 6 results in charging distance of approximately 10 cm to 20 cm, along with the effective charging area of about one square foot (1 ft² or 929 cm²). In this disclosure, the term “approximately” or “approximately equal” means the values are equal within manufacturing and measurement tolerances. For example, approximately 10 cm means the distance equals 10 cm, within variation to account for measurement accuracy, temperature changes, and other factors that may affect the value.

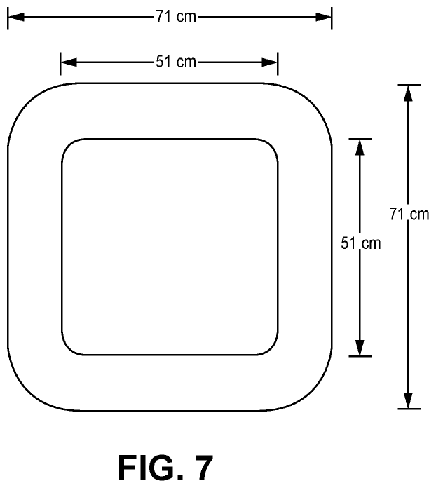

FIG. 7 is a conceptual diagram illustrating an example primary coil of this disclosure. The example of FIG. 7 is one example implementation of the primary coil of this disclosure as described above in relation to FIGS. 1, 3, 4C, and 6. The charging system of this disclosure may use a coil as described in FIGS. 6 and 7, or a primary coil of a different geometry, depending on the application. For example, the coil of FIGS. 6 and 7 may be implemented in system 120 of FIG. 4C, in the example of a mattress. In other examples, the primary coils of system 120 may have a more rectangular geometry with different dimensions, such as to set a charging distance of less than ten centimeters for use in a chair pad, vehicle seat pad, a wearable recharging system, and so on.

The square coil geometry in the example of FIGS. 4C, 6, and 7 may provide a flat distributed magnetic field strength as shown in FIG. 6, in contrast to a circular coil that provides a strong point concentrated paraboloid magnetic field. The example simulations of FIG. 6 resulted from a square transmitting coil with inner dimensions of 51 cm by 51 cm and outer dimensions of 71 cm by 71 cm which is shown in FIG. 7, which resulted in a charging distance of 10 cm to 20 cm.

The circuitry driving the one or more coils of system 120 depicted in FIG. 4C that are similar to the coils in the examples of FIGS. 6 and 7 may include one or more resonant capacitors paired with each coil. The capacitors, along with the inherent self-inductance of the primary coils Tx coils, may result in resonant tank associated with each of coils 124 to resonate at a selected predetermined center frequency. Adjusting the type of wire, number of coils, size, shape and so on of the coils may adjust the selected center frequency.

For example, Litz wire, named after the German term litzendraht essentially meaning “woven wire”, is constructed with multiple individually insulated strands of magnet wire. Litz wire may provide flexibility for coils 124 of FIG. 4C, and may reduce losses due to skin effect and proximity effect. One factor for choosing what type of wire to use involved alternating current (AC) and the skin effect. When AC runs through a conductor, the current density is heavily distributed near the surface of the coil. Litz wire insulates the individual wires in a conductor so that the AC current has a much larger surface area to travel through, thereby reducing the losses due to the skin effect.

In some examples, the wire used to implement the primary coils may comprise an extruded sheathing, which may be desirable for dielectric break-down, water resistance, and durability. Other types of wire, number of turns, coil geometry and so on may impact the selection of driver circuitry.

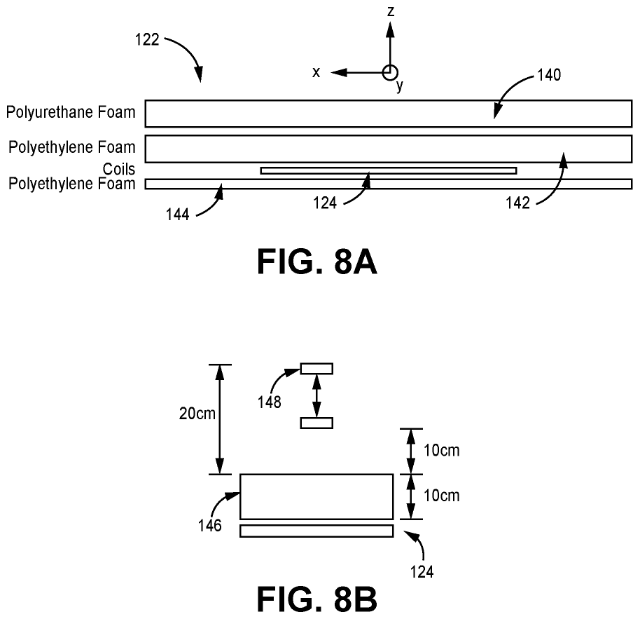

FIGS. 8A and 8B are conceptual diagrams illustrating an example separation pad for the electric (E) and magnetic (H) field protection in recharge mattress pad according to one or more techniques of this disclosure. Pad 122 and coils 124 of FIGS. 8A and 8B are examples of pad 122 and coils 124 described above in relation to FIG. 4C.

Implementation of the power transmitting system of this disclosure, e.g., system 120 of FIG. 4C, into a mattress pad may be desirable for several reasons. First, the average person may sleep approximately six hours a day, which may give the system ample time to recharge the battery while integrating recharging more conveniently into the user's everyday life. In other words, recharging the neurostimulator could be as simple as going to bed.

Secondly, the design of the mattress pad itself can be beneficial to both the user and the transmitting coil. In some examples, the mattress pad may include a removable cover, a top layer of polyurethane 140 for comfort, and a bottom support layer 142 made of polyethylene 142 for support. In the example of FIGS. 8A and 8B, transmitting coil 124 is placed inside a bottom polyethylene foam layer 144.

In some examples support layer 142 may include substantially planar top surface and a substantially planar bottom surface opposite the substantially planar top surface arranged parallel to the plane of the transmitting coil or coils 124, as shown in the example of FIG. 8A. The plane of the transmitting coil is in the plane defined by the X-Y axis. In other words, defined by a first axis and a second axis perpendicular to the first axis, wherein the single plane includes the first axis and the second axis. Any of layers 140, 142 and 144 may be made from a material with a predetermined resiliency. Some layers may be stiffer and less subject to deformation when a load is on the layer, such as a patient laying on mattress pad 122. In some examples, support layer 142 may be configured to support a mass of a user, e.g., patient 12 of FIG. 1, and maintain a predetermined spacing between the plane of the power transmitting antenna 124 and the user during compression of the material from the mass of the user. In some examples, both layers 140 and 142 may be considered the support layer and maintain spacing between the user and coils 124. In some examples, the resiliency of layer 140 is substantially the same as the resiliency for layer 142. In other examples, layer 142 may have first resiliency and layer 140 may be a second material with a second resiliency.

In some examples, surrounding the mattress, a cover, made from a blend of polyester and cotton (not shown in FIGS. 8A and 8B) may provide a soft outer layer that would not distract from the comfort of the foam padding inside. Additionally, a polyurethane backing may block fluids, dust mites, and allergens. Transmitting coil(s) 124 would have a base layer 144 of polyethylene below with a thicker layer (142 and 146), e.g., approximately 10 cm, above. Base layer 144 may also include substantially planar top surface and a substantially planar bottom surface opposite the substantially planar top surface arranged parallel to the plane of coils 124. The power transmitting antenna of coils 124 may be sandwiched between base layer 144 and the support layer 142, as shown in the example of FIG. 8A. The properties of the mattress foam may be beneficial in protecting the coil from plastic deformation, as well as creating a buffer distance between the user and the electric and magnetic fields of the transmitting coil.

As described above in relation to FIG. 6, spacing the patient further away from the coils, may result in a lower drop-off of magnetic field strength at distances of 10 cm and 20 cm from the surface of the pad, as shown in FIG. 8B. In other words, the mattress pad design was created to ease the burdensome process of recharging and provide greater comfortability for the user, create a protective layer for the transmitting coil, and act as a buffer between the user and the harmful electric and magnetic fields.

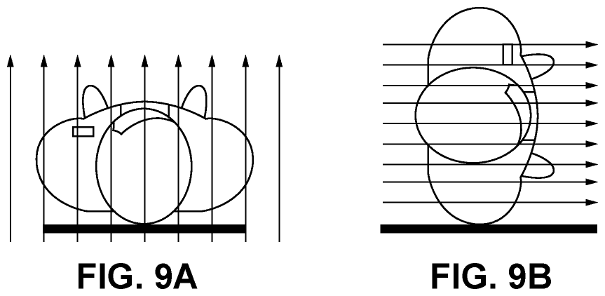

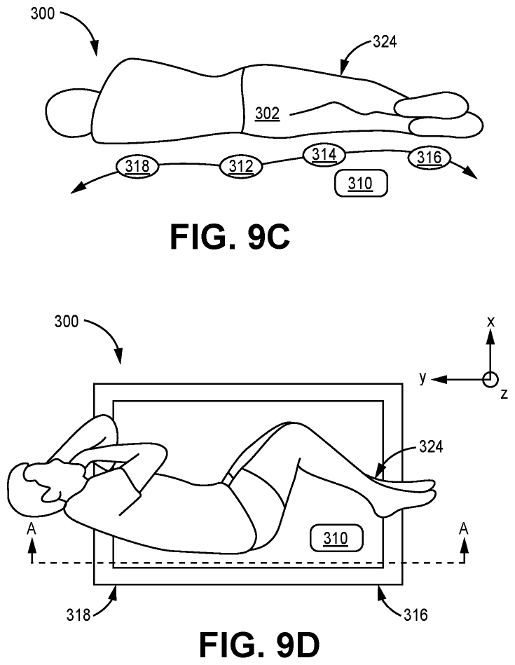

FIGS. 9A, 9B, 9C, and 9D are conceptual diagrams that depict a patient's sleeping positions when using the wireless power transfer system of this disclosure. Incorporating the recharging process into the patient's sleep cycle, the implant's position and orientation in respect to the transmitting coils may be more predictable. A user is likely to sleep on their back, front, right side or left side on a mattress. Regarding how this influences the implant's position and orientation, it can be expected that the implant 148 may will be parallel to transmitting coil(s) 124, as shown in FIG. 9A, as well as above in FIGS. 8A and 8B. In other examples, implant 148 may be oriented at a different angle based on the position of the patient, such as perpendicular to transmitting coil(s) 124 as shown in FIG. 9B. In this way, the wireless power transfer system may only need to provide a magnetic field in two of the three magnetic field directions. In other words, the patient will likely be sleeping along the long axis of the bed, rather than standing up in the z-direction, or rotating to sleep along the short axis of the bed.

FIGS. 9C and 9D are conceptual diagrams illustrating an example wireless power transmitting system including multiple charging coils, according to one or more techniques of this disclosure. System 300 is an example of systems 100 and 120 described above in relation to FIGS. 1 and 4C and may have the same or similar functions and characteristics as systems 100 and 120. FIG. 9C represents an A-A sectional view of FIG. 9D.

FIG. 9C illustrates patient 324 with power receiving device 302 lying on a flexible wireless power transmitting system 300 including coils, depicted by 312, 314, 316 and 318, and driver circuitry 310. Power transmitting system 300 may generate one or more electromagnetic fields to wireless transfer power to power receiving device 302. In some examples, power receiving device 302 may be an implantable medical device, such as IMD 220 of FIG. 2. In other examples, power receiving device 302 may be a wearable medical device, or other portable electronic device, such as a mobile phone, music player, fitness tracker and so on. Though shown as implanted in the buttock area, or located in a hip pocket of patient 324, in other examples, power receiving device 302 may be located in the pectoral area, near the clavicle, ankle, or any other location on patient 324.

The example of FIGS. 9C and 9D depict power transmitting system 300 as a mat or bed in which patient 324 is positioned. In other examples, power transmitting system 300 may be installed in a chair, the seat of an automobile, or other vehicle, and other similar locations. FIG. 9C is an example cross section view and FIG. 9D is a top view of a charging coil system 300 of this disclosure. Coil cross-sections 312, 314, 316 and 318 may be implemented as multiple loops of a single coil, in some examples. In other examples, coil cross-sections 312, 314, 316 and 318 may depict separate overlapping coils, or separate coils that do not overlap.

Similar to the description above with respect to FIGS. 1 and 2, driver circuitry 310 may include processing circuitry, coil driving amplifier circuits, tuning circuits, communication circuits, sensing circuitry including filters, amplifiers and so on connected to one or more sensors, along with other circuitry not specifically listed (not shown in FIG. 3). Driver circuitry 310 may drive each of the one or more coils of system 300 together or separately. As described above in relation to FIG. 2, the processing circuitry may control the phase angle, frequency, magnitude, and other characteristics of the driving signal to focus, or to broaden, the electromagnetic field generated by the one or more coils.

In some examples, system 300 may include a single coil, as shown in the example of FIG. 9D. In other examples, system 300 may include multiple coils as shown in FIG. 4C. In some examples, system 300 may include a first coil near the upper body of patient 324 formed by 312 and 318, along with a second coil near the legs of patient 324 formed by 314 and 316. In other examples, system 300 may include overlapping coils, e.g., a first coil formed by 314 and 318 near the upper body of patient 324 and a second coil formed by 312 and 316 near the lower body of patient 324. In other examples, system 300 may include three or more charging coils, which may be arranged as overlapping or adjacent coils. The portions of the coils indicated by 312 and 314 are not shown in FIG. 9D.

The spatial relationship between driver circuitry 310 and the coils may change as patient 324 moves. Patient 324 will likely be sleeping along the long axis of the bed, rather than standing up in the z-direction, or rotating to sleep along the short axis of the bed, as shown in FIG. 9D. In other words patient 324 may usually lie on the xy-plane, laying head-to-feet along the y-axis. A benefit of integrating the charging process into a user's sleep cycle is that the magnetic field in the y-direction may become statistically ignorable. The IMD 302 may be such that the receiving coil, e.g., secondary coil 16 and secondary coil 40, depicted in FIGS. 1 and 2 may be oriented normal to the surface of the body in the abdominal, lower-back, or upper pectoral regions of the body. The orientation, along with the normal sleeping patterns of an adult where lying on their front, back, side, or at an angle between, means that the additional magnetic flux due to the y-direction magnetic fields may negligible. FIG. 9A shows the z-direction (normal) and FIG. 9B the x-direction (parallel) magnetic fields based on the primary coils described above in relation to FIGS. 6 and 7.

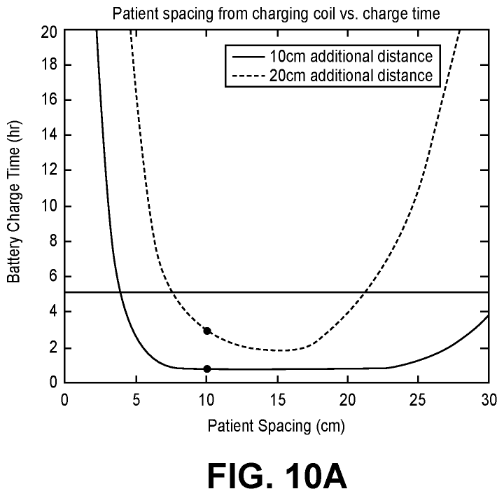

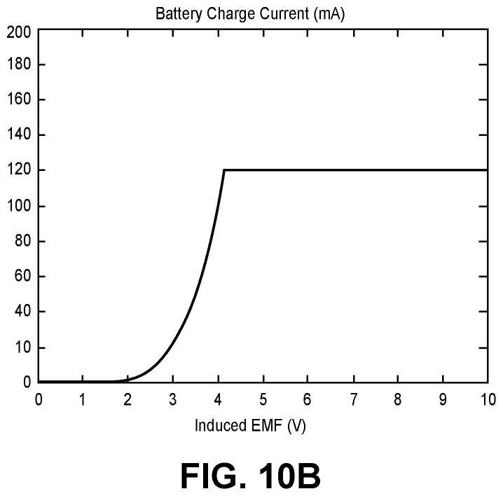

FIG. 10A is a graph illustrating the impact on charge time from changes in the spacing between a primary coil and a secondary coil. The example of FIG. 10A helps to describe the physics behind induction charging and what factors that will affect the strength of the energy transfer. Induction charging occurs when an AC current runs through a transmitting (Tx) coil, e.g., primary coils 124 described above in relation to FIG. 4C, and produces a changing magnetic field, as shown in FIG. 4A. This magnetic field can be derived from differently shaped coils using the Biot-Savart Law, which can be seen in Equation 1.

$$\\ \begin{matrix}d \vec{B} = \frac{{μ}_{o}}{4 π} \frac{I \vec{dl} \times \hat{r}}{{r}^{2}} & ( 1 )\end{matrix}$$

In this equation, d\vec{B} is the magnetic field vector due to a current element, I\vec{dl}. The equation takes into account the permeability of the surrounding space, so as well as the distance between the point and the current element as well as the vector direction between them, \hat{r}.

That changing magnetic field flows through the secondary receiving (Rx) coil and creates a changing magnetic flux, which induces an electromotive force (EMF) across the Rx coil's windings. The magnitude of this EMF can be calculated using Faraday's law in Equation 2.

$$\\ \begin{matrix}\vert ε \vert = \frac{d {Φ}_{B}}{dt} = \frac{d}{dt} \int B ( t ) \cdot dA & ( 2 )\end{matrix}$$

In this equation, the induced emf, ε is equal to the rate of change of the magnetic flux going through the secondary coil,

$$\\ \frac{d {Φ}_{B}}{dt} .$$

This EMF causes a voltage potential to rise across the windings that induces a current. The amount of EMF voltage created is also directly related to the mutual inductance of the two coils and is shown in Equation 3 below.

$$\\ \begin{matrix}{ε}_{m} = M ( \frac{{dI}_{1}}{dt} ) & ( 3 )\end{matrix}$$

In this equation, ϵₘ is the EMF voltage, M is the mutual inductance, and dl₁ is the current in the first coil. Since the mutual inductance is such a crucial determinant of the power transfer, a deeper dive into it was needed. From a mathematical standpoint, Equation 4 shows how to calculate mutual inductance.

M=k\sqrt{L₁L₂} (4)

Above, L₁ and L₂ are the inductances of the two coils. Inductance is defined as the amount that an electrical conductor opposes the change in the electric current flowing through it. In addition, k is the coupling coefficient of the two coils. The coupling coefficient of induction coils is the ratio of the magnetic flux was transferred from one coil to another to the total magnetic field produced by the transmitting coil. An inductive coil may be measured using Equation 5 where N is the number of turns, w is the wire diameter, Dₒ is the outer diameter, and p is the spacing in between each turn.

$$\\ \begin{matrix}L = \frac{{{N}^{2} ( {D}_{o} - N ( w + p ) )}^{2}}{16 {D}_{o} + 28 N ( w + p )} ( \frac{39.37}{{10}^{6}} ) & ( 5 )\end{matrix}$$

These results of the coupling coefficient (k) and the previously calculated inductances could be used in Equation 2 to find the mutual inductance of the two coils.

In three-dimensional space, solving for these magnetic fields yields three equations for the magnetic field in the z-direction, y-direction, and x-direction, as described above in relation to FIGS. 9C and 9D. A noted above, the patient may lie on the xy-plane, laying head-to-feet along the y-axis. A benefit of integrating the charging process into a user's sleep cycle is that the magnetic field in the y-direction becomes statistically ignorable. Based on implant location and sleeping patterns of an adult lying on their front, back, side, or at an angle between, means that the additional magnetic flux from y-direction magnetic fields may be negligible.

In some examples, the rate at which the induced EMF induced in the secondary coil drops off as distance is increased from the coil for variously different shaped coils. There may be a sharp drop-off when the secondary coil first starts being spaced away from the coil. In this manner, the size, shape, materials and so on for the primary coil may be determined for a variety of wireless power transfer applications. For the mattress application, described above in relation to FIGS. 8A-9D, the 51 cm square Tx coil was the most effective at a distance of 20 cm, and a 71 cm square Tx coil was the most effective at a distance of 30 cm. As noted above, differences in wire size, type, materials and so on may result in variation in spacing distance.

The estimated charge time can be estimated by using Equation 6.

$$\\ \begin{matrix}Charge Time ( h ) = 0.94 * \frac{Battery Capacity ( mAh )}{Battery Charge Current ( mA )} & ( 6 )\end{matrix}$$

The battery charge current can be found from the induced EMF using Equation 7.

Battery Charge Current (A)=(0.16592*EMF(V))^{5.495} (7)

FIG. 10B is a graph illustrating an example battery charge current limit in the power receiving device. In some examples, the power receiving device may include protection circuitry, e.g., to limit charging current, such as from recharge module 38 to power source 18, as described above in relation to FIG. 2. In the example of the IV curve of FIG. 10B, circuitry inside an example power receiving device may protect the battery by keeping the battery charge current from exceeding 120 mA. In other examples, the current limit may be higher or lower and may depend on factors such as: battery size, battery type, battery capacity, charging circuitry arrangement and so on.

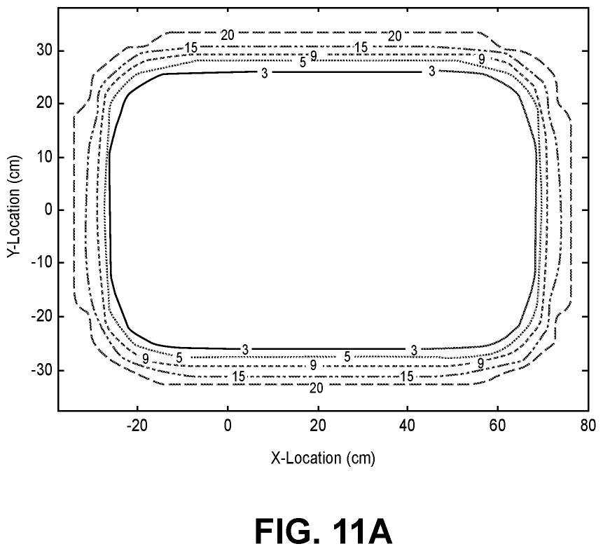

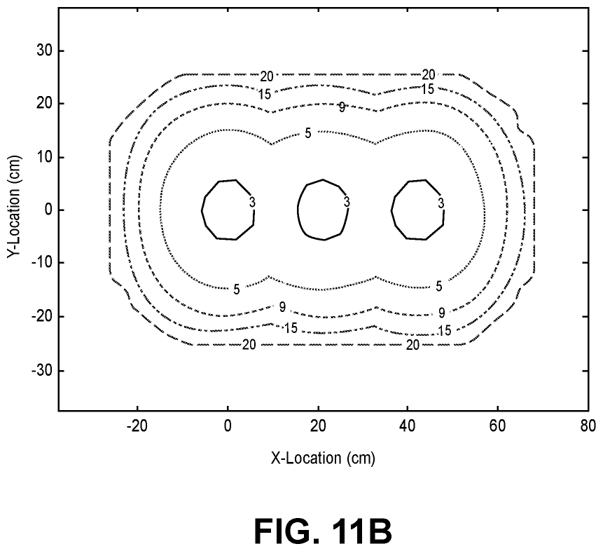

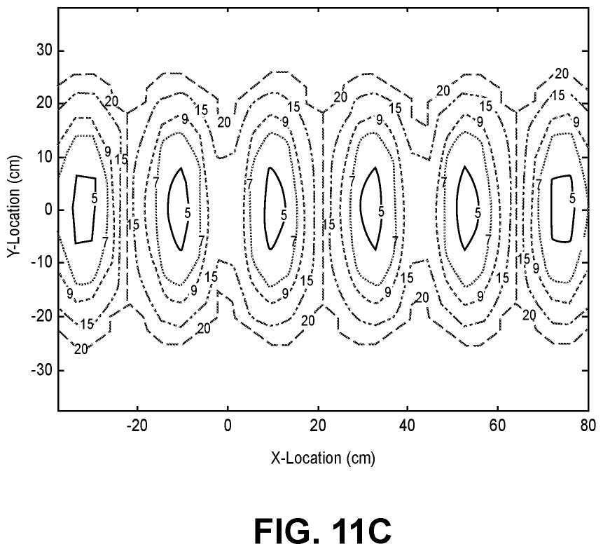

FIGS. 11A, 11B and 11C are conceptual diagrams illustrating simulated charge time contours for an implantable medical device being charged by a mattress pad based wireless power transfer system. The charge times contours shown in FIGS. 11A, 11B and 11C are based on a three coil system such as system 120 described above in relation to FIG. 4C. The example of FIG. 11A depicts a contour plot of charge times for a prone patient spaced 10 cm from the surface of the mattress pad, with the battery current limitations described above in relation to FIG. 10B. The charge times are faster for a patient located near the center of the mattress and may increase, e.g., longer charge times, as the patient moves toward the edges of the mattress. The charge times are for a fully discharged battery, and correspond to the magnetic field strength distribution described above in relation to FIG. 6. In the example of FIG. 11A, charge times near the center of the mattress, and therefore near the center of primary coils 124 of FIG. 4C, are calculated to be approximately three hours. Charge times near the edges may increase to twenty hours.

The example of FIG. 11B depicts calculated contour plots for a patient in a prone position where the secondary coil is located 20 cm from the surface of pad 146, and oriented approximately parallel to coils 124 e.g., as depicted in FIG. 8B. For the example of FIGS. 11A and 11B, a patient is lying prone on the mattress, may expect an effective charging area of 5.5 ft² (0.51 m²) at a 10 cm distance from the surface of the spacing pad, and an effective charging area of 2 ft² (1858 cm²) at a distance of 20 cm. Of course, changing the shape, size, materials and so on of the primary coils may change the charging times and effective charging area. As noted above in FIG. 4C, the power receiving unit battery capacity, current, effective secondary coil size and so on may also impact the values calculated for the examples of FIGS. 11A and 11B.

In addition, relative orientation of the primary and secondary coils may also affect the power transfer efficiency, and therefore the charge times. The example of FIG. 11C depicts contour plots of charging times for a power receiving device located 10 cm from the surface of the pad for a patient on their side. Comparison of FIGS. 11B and 11C show that charging times near the center of the pad may increase when the orientation of the secondary coil is not parallel to the primary coil.

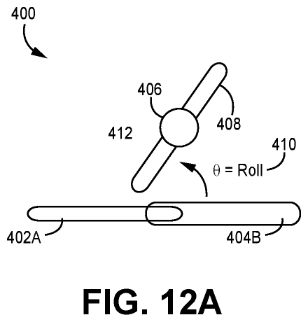

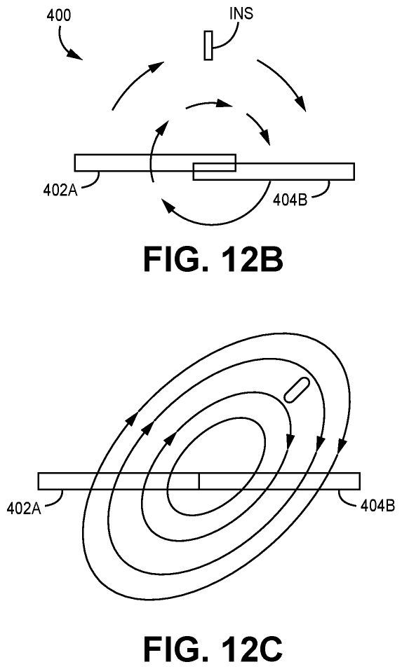

FIGS. 12A, 12B, and 12C are conceptual diagrams illustrating a multiple coil wireless power transfer system configured to compensate for the relative orientation of the primary coil and the secondary coil. In FIG. 12A, system 400 depicts a patient 412 sleeping with a roll angle 410 on a multiple coil mattress charging system, such as system 120 described above in relation to FIG. 4C. Roll angle 410 of patient 412 may place a power receiving device implanted, worn or carried by patient 412 at an angle relative to the primary coils, e.g., coils 124 of FIG. 4C. In the example of FIGS. 12A, 1B and 12C only coil A 402 and coil B 404 are shown. The angle of the secondary coil of the power receiving device may be the same or different from roll angle 410. Though only two coils are shown in FIGS. 12A, 12B and 12C, any number of coils may be active, at different phase angles, to generate an electromagnetic field at the desired angle. For example, in a four-coil system, two, three or four coils may be energized to generate the field as shown in FIG. 12C.

As described above, though the example of FIGS. 12A, 12B, and 12C focus on the example of a medical device, such as IMD 14 described above in relation to FIGS. 1 and 2, system 400 may wirelessly provide power to any compatible device configured with a secondary power receiving antenna, e.g., mobile phone, hearing aid, fitness tracker and so on. Also, as described, system 400 may be implemented as a portable pad that may be placed on a chair, or similar furniture, and may be embedded into a chair, vehicle seat and so on. In some examples, the coil size, shape, number of windings and so on may differ from the arrangement of system 120 described above in relation to FIGS. 4C-11C to adjust for the spacing between the primary and secondary coils, desired charging area, and so on, for wireless power transfer systems implemented in a chair, or other furniture different from a mattress.

As described above, in some examples, a multi-coil array, similar to system 120 of FIG. 4C, may provide directional control of the electromagnetic field by exercising at least two coils simultaneously out of phase with each other. In some examples, the efficiency of wireless energy transfer may improve when the primary and secondary coils are approximately parallel, when compared to being at a relative angle to each other. A multi-coil array may exercise at least two coils simultaneously out of phase with each other, e.g., between 1-180 degrees out of phase to adjust the angle of the magnetic field. In some examples, both the amplitude and phase of the at least two coils may be adjusted to change the field direction. In this manner, the patient could “roll on their side” or “lay at an angle.” As described above in relation to FIGS. 9C and 9D, as long as a patient is positioned vertically along the length of the mattress, the system may adjust the magnetic field angle to account for the patient “roll,” but patient “yaw” and “pitch” may not require adjustment.

Processing circuitry of the multi-coil wireless power transfer system, e.g., processing circuitry 50 of FIG. 3, or any other processing circuitry of FIG. 1, may execute programming instructions to adjust the phase and/or amplitude of the AC current delivered to two or more coils. In some examples the programming instructions may include to identify which two coils are closest to the power receiving unit, e.g., implantable medical device. The processing circuitry may then control the primary coil driving circuitry, e.g., charging module 58 of FIG. 3, to vary the relative phase to change the phase angle between 0-180, and calculate one or more system metrics to determine the desired phase angle. As described above, the one or more system metrics may include a power transfer efficiency, metal loading, heat calculation, receiving an indication from the power receiving device of the magnitude of received charging current, and so on.

In some examples the processing circuitry may determine the desired phase angle with a binary search. In some examples, the processing circuitry may also adjust the relative magnitude of power, e.g., amplitude of electrical current, delivered to the coils, either at the same time or a different time from when adjusting the phase difference between the coils. For example, start the phase angle at approximately 90 degrees, the check 45 degrees and 135 degrees. Based on the calculated system metric(s) select one of 45 or 135 degrees and vary the phase angle by, for example, 45 degrees plus 22.5 degrees and 45 degrees minus 22.5 degrees. The processing circuitry may continue to refine phase angle as needed. A binary search is just one possible example of many possible techniques to vary the phase angle and check system metrics to determine the effective power transfer of the system.

FIGS. 13A, 13B, and 13C are conceptual diagrams illustrating an example return-wrapping technique for transmitting coils in wireless power transfer applications. The examples of FIGS. 13A and 13B describe one possible implementation of the return-wrapping technique for reducing the electric field while maintaining the magnetic field. Other examples may include more or fewer wrapping, different shapes of coils, other connection techniques and so on.

As described above in relation to FIGS. 4A and 4B, a strong magnetic field may improve wireless power transfer, but may also result in a strong electric field also generated by the primary coil. Voltage magnification across the coil when it resonates may develop across the coil is upwards of a thousand volts, e.g., E1 426 and E2 428. High voltage may raise concern for the use of proper insulation, as well as for the emission of a strong E-field as well. IEEE standard C95.1-2019 provides limits that govern the human exposure to electric, magnetic, and electromagnetic fields ranging from 0 Hz to 300 GHz. The electric field, e.g., E1 422, produced by the coil may be estimated by taking the voltage drop from the outermost turn, e.g., turn 1 (430) to the innermost turn, e.g., turn 6 (432) in the example of FIG. 13A, and dividing by the arclength that reaches out a distance to the patient. An E-field, e.g., E-field 112 described above in relation to FIG. 4B may exceed the standard by an order of 10³.

In some examples of inductive primary coils, the coils are wrapped from the innermost turn to outermost. In contrast, the primary coil of this disclosure is instead wrapped so that the turns wrap inwards and then back out again. FIG. 13C shows an example connection between the inward wrapped coil and the outward wrapped coil. In some examples, such as shown in FIG. 13A, the wires for the inward and the outward turns are arranged to be near each other. FIG. 13A is a cross section (424A and 424B) of FIG. 13B. In the twelve turn example of FIG. 13A, the first turn and the last turn are arranged in close proximity to each other (420). Similarly, other turns such as turns two and eleven (421), and the last inward turn six (432) may be located near the first outward turn seven (434).

FIG. 13A shows a cross section of an example return-wound coil and the electric field E1 (422) and E2 (428) produced by each half. The coil is still wound in one direction, so that current only flows in one direction at a time, clockwise or counterclockwise. Therefore, the return-wound primary coil may not compromise the magnetic field. Wrapping a coil using this return-wrapping technique may result in a coil of similar size and that produces the same magnetic field as a traditionally wound coil. One feature of the design is that the electric field generated by the voltage drop across the coil's turns is split in half and set against itself so that it is at least partially nullified. This may enable a user or patient to come in close proximity to the coil, strengthening the magnetic field seen by the implanted medical device and increasing the charge speed.

In this manner the E-field, E1 (422), produced by the inwardly wrapped half of the coil may be approximately equal and opposite to the E-field produced by the outwardly wrapped half of the coil, E2 (428). In this manner, E1 (422) may substantially cancel E2 (428) via superposition. In some examples, E1 (422) may exactly equal E2 (428) and completely cancel out E2 (428). In other examples, because of limitations of manufacturing, assembly, location of wires, imperfections in coiling, wire diameter, insulation and so on, E1 (422) may only approximately equal E2 (428) and the E-fields may therefore substantially cancel each other. In some examples, the return wrapping technique of this disclosure may nearly cancel out the near-field electric field generated while having little to no effect on the magnetic field or effectiveness of the recharging process.

FIGS. 13D and 13E are graphs depicting an example of an E-field generated by primary coils. The example of FIG. 13D depicts a traditionally wrapped coil, e.g., outward to inward, at a phase angle of zero (0°). The example of FIG. 13E depicts a return-wrapped coil according to one or more techniques of this disclosure, and as depicted in FIGS. 13A-13C at a phase angle of zero (0°). In the example of FIG. 13E, the E-field appears to be substantially canceled, when compared to FIG. 13D. The return wrapping method appears to have reduced the strength of the E-field. Both images use a logarithmic scale going from 1 to 2000 V/m.

FIGS. 13F and 13G are graphs depicting an example of an H-field that may be generated by primary coils. The example of FIG. 13F depicts the magnetic field of a traditionally wrapped coil, e.g., outward to inward, at a phase angle of zero (0°). The example of FIG. 13G depicts a return-wrapped transmitting coil as depicted in FIGS. 13A-13C at a phase angle of zero (0°).

The graphs in the examples of FIGS. 13F and 13G depict the strength of the magnetic fields of both wrapping methods, using a scale of 1 to 500 A/m. The strength of the magnetic fields, and ultimately the amount of transferable power, appears to be nearly identical. In the manner, the simulations modeled by FIGS. 13D, 13E, 13F, and 13G show the new return wrapping method may reduce the electric field, while maintaining the magnetic field. Thus, the return-wrapping techniques of this disclosure may improve patient safety as well as allow for more design flexibility when compared to other techniques.

Some other example solutions to reduce the exposure of a user to the E-field may include the distribution of capacitors throughout the coil. In some examples, adding capacitors to the primary coil may reduce the overall electric field due to the voltage magnification of series resonance. However, adding capacitors for each turn, e.g., four per turn may increase cost and complexity of such a primary coil, and may reduce reliability and robustness, which may be desirable for a primary coil in a flexible pad, such as a mattress or a seat.

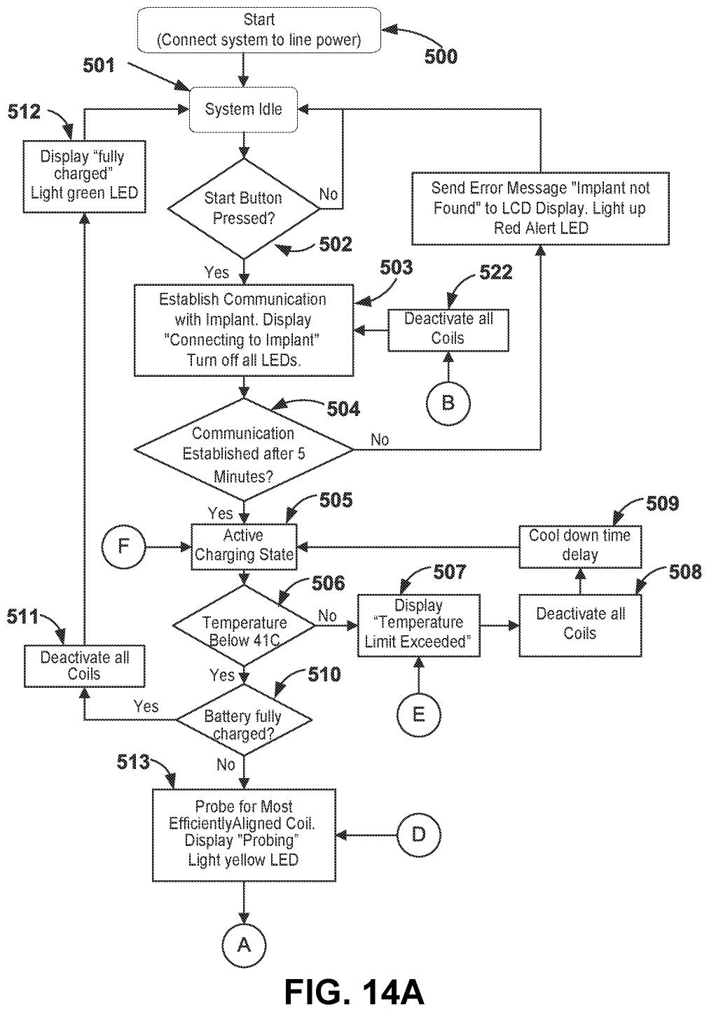

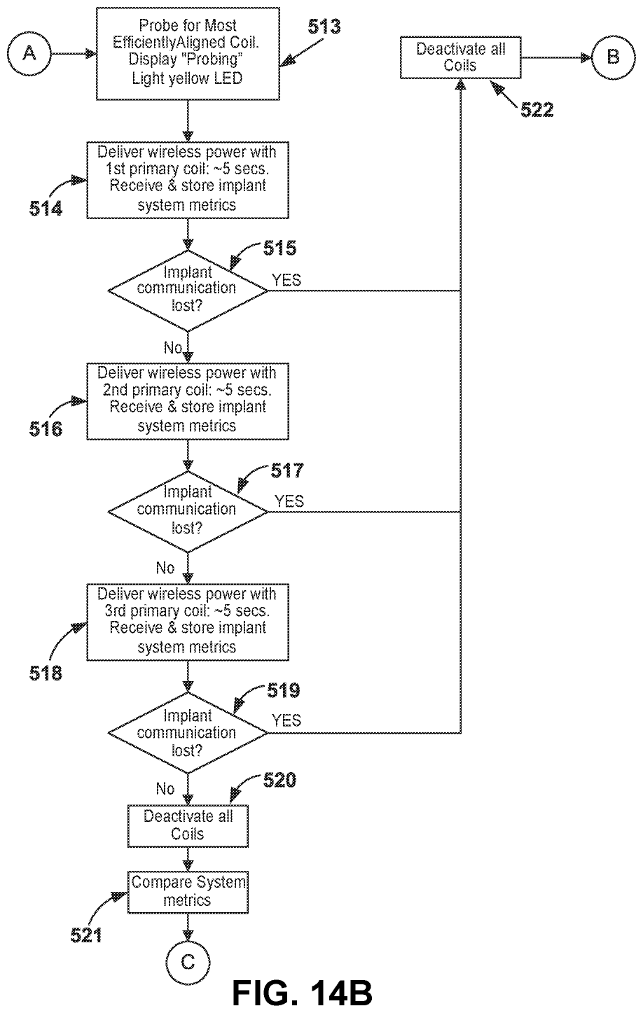

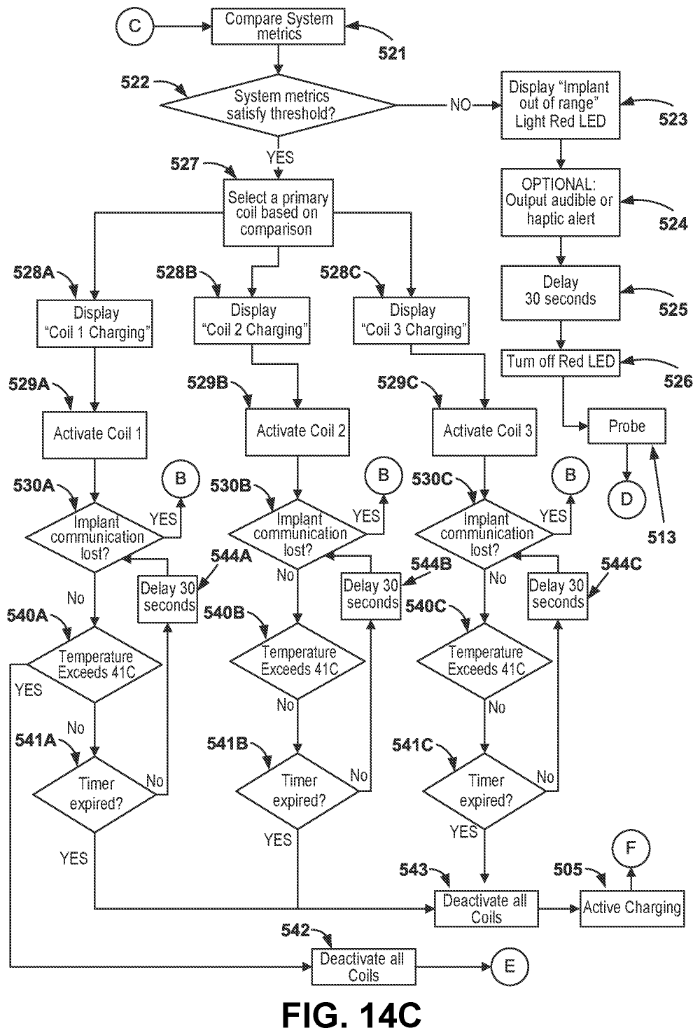

FIGS. 14A, 14B, and 14C describe one example operation of the wireless power transfer system of this disclosure. In the example of FIGS. 14A-14C, processing circuitry of the wireless power transfer system, e.g., any one or any combination of processing circuitry described above in relation to FIG. 1, may execute the steps of FIGS. 14A-14C to activate a single primary coil of a multi-coil system, e.g., similar to system 120 of FIG. 4C. In other examples, the processing circuitry may activate two or more primary coils to adjust the angle of the magnetic field, as described above in relation to FIGS. 12A-12C.

The software executed by processing circuitry of the system, in the example of FIGS. 14A-14C may operate in three stages as outlined. For the first stage, check communication with the power receiving device. If communication not established, do not begin charging. In one example, the second probing stage may include to activate each coil independently, e.g., probing for the effect of each coil by using the following steps:
1) Activate a first coil
2) Check charge current or other system metrics in energy receiving device, e.g., an implantable medical device
3) Deactivate first coil
4) Activate second coil
5) Check current in in energy receiving device
6) Deactivate second coil
N1) Activate Nth coil
N2) Check current in the power receiving unit
N3) Deactivate Nth coil

Compare the determined system metrics and for the third stage activate the most effective coil, e.g., begin charging with coil which produced largest charge current. During the predetermined charging duration, check temperature and communication with implant periodically, e.g., every 30 seconds. If temperature gets too high above a temperature threshold, then stop charging or reduce power. The processing circuitry in some examples may include a PID controller if power receiving unit and or power transmitting pad have temperature sensor(s). If at any point, communication is lost, start algorithm over, and finally stop charging when at full capacity.

In more detail, for the first stage, the processing circuitry may establish communication with the implant as shown by FIG. 14A. If communication is established, the system may begin the second stage and probe for the location of the power receiving device by activating each transmitting coil individually and measuring the response from the receiving device, e.g., IMD 14 of FIGS. 1 and 2. In the example of FIG. 14A, the processing circuitry may also perform a temperature check to verify that the implant temperature is less than 41° C. e.g., by receiving a signal from temperature sensor 39 of FIG. 2. In other examples, the system processing circuitry may perform a heat calculation as described above in relation to FIGS. 1, 2, and 12A. In some examples the processing circuitry may check the charge state of the implant battery before starting the probing stage, as described above in relation to FIG. 2.

The example of FIG. 14B depicts one example of a probing procedure. The processing circuitry may deliver wireless power from each primary coil in turn, e.g., one at a time. The processing circuitry may determine one or more system metrics, such as power transfer efficiency, battery current delivered to the battery of the implant, metal loading, calculated heat, and so on for each primary coil. The processing circuitry may compare the one or more system metrics and select one primary coil of multi-coil system with the relative position and relative orientation to the secondary coil of the implant to deliver the most effective wireless power transfer. The most effective wireless power transfer may be based on the highest efficiency, the most battery current delivered or any other system metrics, or combination of system metrics. In some examples, such as a loss of communication with the implant, the processing circuitry may deactivate all the coils and try to reestablish communication, e.g., as shown in FIG. 14A.

In the third stage, as shown in the example of FIG. 14C may activate the selected transmitting coil for a predetermined duration, e.g., five minutes, ten minutes, fifteen minutes, and so on. During the predetermined charging duration, the processing circuitry may also periodically check the response from the implant, e.g., every 30 seconds or so, to protect against overcharging and overheating. In some examples, any of the durations, e.g., charging duration, checking period, etc. may be selectable by a user via the user interface of the remote control depicted in FIG. 5, or some other user interface of system 100 depicted in FIGS. 1, 2 and 3.

In some examples, the processing circuitry may set a timer for the predetermined charging duration. After the preselected duration, e.g., of ten minutes, if the implant is not fully charged, the system may return to the active charging state, and again start the probing stage to check for any changes in relative location between IMD 14 and the selected primary coil. The programming instructions stored at a memory location of the system may cause the processing circuitry to check the temperature of IMD 14. If the temperature exceeds 41° C. at any time in the process, the system may enter a predetermined cooldown phase where no transmitting coil is powered, and the implant can cool down. In some examples the time delay for the cooldown phase may be fifteen minutes, ten minutes, or some other duration. The programming instructions may also include checks for other errors during the charging process, causing the processing circuitry to react, such as to prompt the user via a user interface display, e.g., as described above in relation to FIG. 5. Throughout the charging process, the processing circuitry may display the current state and status with the user interface, e.g., the display 132, LED lights 134 and so on, depicted in FIG. 5.

The charging systems described herein may help increase the charging distance from 3 cm to 10-20 cm, have an effective charging area of 1-3 ft², and incorporate the recharging process into the user's everyday activities that could be completed in under five hours. By expanding the dimensions of the coil, the target charging range can be increased. As described above in relation to FIG. 6, square a 51×51 cm coil, with about 70 turns, may generate a magnetic field for 10 cm distance, e.g., from the inner dimensions of the transmitting coil, and reach the 20 cm target distance with the outer dimensions of the transmitting coil. Both target distances of 10-20 cm would include an additional 10 cm buffer zone that would protect that the user from harmful electric and magnetic fields. This extra space may be desirable to allow the driving circuitry to deliver a higher current to more efficiently charge the battery. Additionally, multiple coils may cover a larger area and account for any changes in the orientation of the patient and implant.

This disclosure described details of a mattress pad system, such as system 120 of FIG. 4C, however, several other examples described herein for integrating the recharging process into the user's everyday life. With the mattress pad example, the user clicks the on button and rolls into bed. No part of their daily routine is hindered and sleeping gives the recharging system a 6 to 8-hour window to charge the battery. Other examples may include a wireless remote and a smaller buffer zone to avoid large mattress pads being added on top of beds.

In more detail, FIG. 14A begins by connecting the system to line power (500)), which may start the system in an idle state (501). When the start button, e.g., power button 130 of FIG. 5, processing circuitry of the system may establish communication with a power receiving device within range of one or more primary coils, or via telemetry module 56 of FIG. 3 (503). The processing circuitry may also cause user interface. e.g., display screen 132 of FIG. 5, to display a message e.g., “connecting to implant,” or similar message, and to turn off indicator lights 134.

If communication is not established after a predetermined period, e.g., five minutes, the processing circuitry may output a message to the display screen, such as “implant not found” and light a red indicator light (NO branch of 504) and enter the idle state (501). Otherwise, if communication is established (YES branch of 504), the system may enter the active charging state (505).

The processing circuitry may verify that the measured or calculated IMD temperature remains less than 41° C. (506). If the temperature rises above 41° C. (NO branch of 506), the processing circuitry may cause the display to notify the user (507), e.g., “temperature limit exceeded.” The processing circuitry may deactivate all coils (508) and start a cool down time delay timer (509) before entering the active charging state again (505).

The processing circuitry may check for the battery charged status, and if fully charged (YES branch of 510), may deactivate all coils (511), cause the display to indicate “fully charged” and may illuminate a green indicator light (512). For a battery that still needs charging (NO branch of 510), the processing circuitry may start the probing process, described above to find the most efficient coil, e.g., of coils 124 depicted in FIG. 4C (513). The processing circuitry may cause the display to indicate “probing” and may illuminate the yellow indicator light.

The example of FIG. 14A describes the probing process in more detail. In the example of a three coil system, the charging module may deliver wireless power with a first coil, e.g., for a few seconds, as described above (514). The processing circuitry may store and implant system metrics for the first coil.

While a communication session is still active, e.g., communication is not lost with the wireless power receiving device (No branch of 515), the charging module may deliver wireless power with a second coil, e.g., for a few seconds, as described above (516). The processing circuitry may store and implant system metrics for the second coil.

While a communication session is still active (No branch of 517), the charging module may deliver wireless power with a third coil, e.g., for a few seconds, as described above (518). The processing circuitry may store and implant system metrics for the third coil. While a communication session is still active (No branch of 519), the processing circuitry may deactivate all the coils (520) and compare the stored system metrics (521). If communication is lost during the probing session, (Yes branch of 515, 517 or 519), the processing circuitry may deactivate all the coils (522) and attempt to reconnect to the power receiving device (503).

The processing circuitry may verify whether any system metrics satisfy a threshold. e.g., charge current, casing temperature, power transfer efficiency, calculated heating and other similar metrics (522). For one or more system metrics that do not meet a predetermined threshold (NO branch of 522), the processing circuitry may cause the “implant out of range” display and may illuminate the red indicator light (523). In some examples, the system may output an audible and or haptic alert (524), delay for a preset time (525), such as for 30 seconds, and turn off the red indicator light (526). Then processing circuitry may restart the probing process (513).

When the system metrics satisfy a threshold (Yes branch of 522), the system may select one of the plurality of charging coils based on the comparison (527). The example of FIG. 14C depicts a three-coil system example. The system may select either coil 1, coil 2 or coil 3, depending which demonstrated the best charging features. Processing circuitry may display one of coil 1 charging (528A, 529A), coil 2 charging (528B, 529B) or coil 3 charging (528C, 529C) and activate the appropriate coil.

If communication with the power receiving device is lost (YES branch of 530A, 530B or 530C) the system may deactivate the coils (522) and may try to establish communication again (503). While the communication session is active (NO branch of 530A, 530B or 530C), the processing circuitry may ensure the IMD temperature does not exceed than 41° C. (540A, 540B or 540C). If the temperature rises above 41° C. (YES branch of 540A, 540B or 540C), the processing circuitry deactivate all coils (542) may cause the display to notify the user (507). e.g., “temperature limit exceeded.”

While the IMD temperature remains less than the temperature limit (NO branch of 540A, 540B or 540C), the system may charge the wireless power receiving device for a preset period of time, e.g., approximately ten minutes. During charging, while the timer has not expired (No branch of 541A, 541B and 541C), the processing circuitry may delay, e.g., for thirty seconds (544A, 544B or 544C) and periodically check for implant communication (530A, 530B or 530C). When the preset period of time expires (Yes branch of 541A, 541B or 541C), the processing circuitry may deactivate all coils 543 and return to the active charging state 505, which may include verifying which coil is providing the best power transfer.

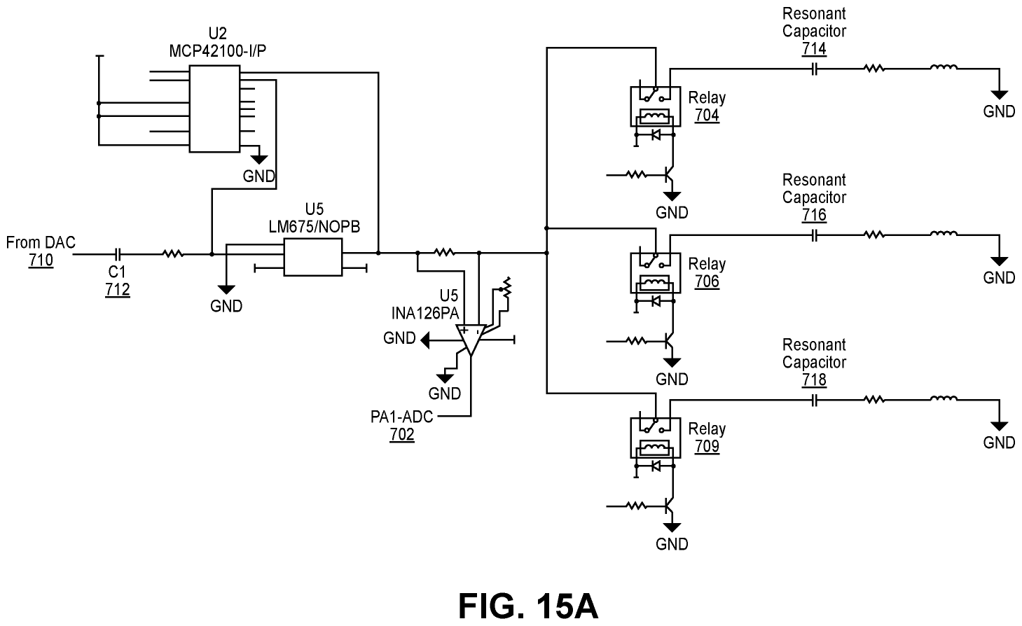

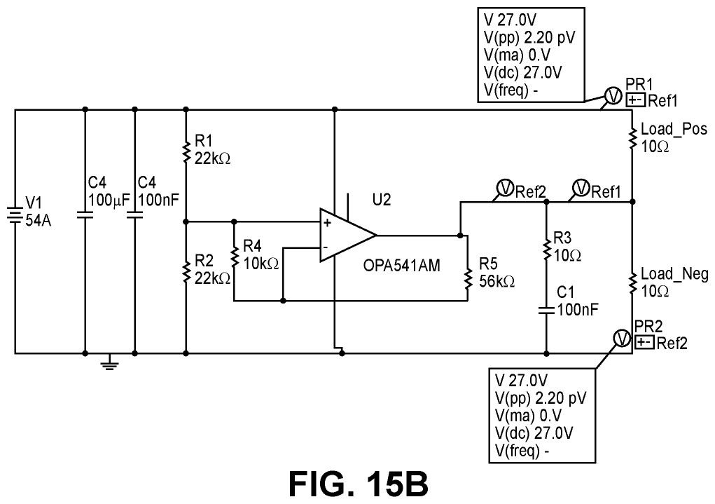

FIG. 15A is a schematic diagram illustrating a portion of the driving circuitry for an example wireless power transfer system. The example circuit in FIG. 15B may be located, for example, with driver circuitry 126 described above in relation to FIG. 4C. The example power supply circuit is one possible implementation of a circuit to control power to multiple primary coils, such as system 120 of FIG. 4C. The component values and circuitry arrangement in the example of FIG. 15A is just one possible example. In other examples, different arrangements, e.g., addition relays for additional coils, and different component values may be used based on the specific implementation, e.g., coils in a mattress bad, a portable charging blanket, and so on.

The circuitry in the example of FIG. 15A drives current through the Tx coils. An onboard digital analog converter (DAC) of a microcontroller (not shown in FIG. 15A) generates an adjustable-frequency, 3.3 V_{PP} sinewave with a 1.65 V DC offset (710) and is then AC coupled by a 1 mF capacitor (712). In the case that the coil bends under the weight of the user, which may change the coil inductance and pushing the tank out of resonance, the frequency can be adjusted through software.

This signal is then amplified by an inverting amplifier consisting of a LM675/NOPB power op-amp and a MCP42100-I/P digital potentiometer to control the gain of the amplifier. SPI communication handled by the microcontroller controls the digital potentiometer. The LM675/NOPB power op-amp is configured to amplify the sinusoidal DAC output from the STM32F4 microcontroller. The gain of the inverting op amp can be controlled through a 100 kΩ digital potentiometer through I²C. The current of the signal is read through a 10 mΩ sense resistor and an INA126PA instrumentation amplifier and read by the microcontroller's ADC input.

The signal then goes through a 10 mΩ sense resistor (R4) read by an INA126PA instrumentational amplifier. The feedback is sent to an onboard ADC (702) and is used to make sure the current through the Tx coils is 2.09 Aₚₖ.

From there, the signal goes through one of three 12 V relays 704, 706, 708 to one of three transmitting coils, e.g., coils 124 of FIG. 4C, and its resonant capacitor. For peak power transfer, each antenna coil may be tuned for resonance at the desired frequency using series capacitance provided by resonant capacitors 714, 716, and 718. In some examples, selection of the resonant capacitor for the specific coil may increase the quality-factor, and minimize the bandwidth of the resonator, thereby reducing spurious emission.

The three relays are controlled by the microcontroller. In some examples, only the relay for the activated is closed so the other coils do not inductively couple with the activated coil and add to the load. In other examples, the processing circuitry of the system. e.g., the microcontroller, may activate more than one coil as described above in relation to FIGS. 12A-12C. The linear amplifier arrangement of the example of FIG. 15A may simplify the driving circuitry and produce a cleaner sinusoidal output waveform when compared to other arrangements. In comparison to using an H-bridge, or square wave producing timer-circuit, by driving the coils with a purer sinusoidal signal as generated by the onboard DAC of the STM microcontroller, noise from the source may be reduced.

FIG. 15B is a schematic diagram illustrating an example power supply for a wireless power transmitting system. The example power supply circuit in FIG. 15B may be located, for example, with driver circuitry 126 described above in relation to FIG. 4C. The example power supply circuit is one possible implementation of a circuit to provide power for the complete system, such as system 120 of FIG. 4C to operate and recharge the implantable device, or other power receiving device.

In accordance with the Collateral Standard IEC 60601-1-11, all medical devices intended for home use shall fall under the Class II designation. Class II designated power supplies allow connection to standard ungrounded NEMA 1-15 (two prong) receptacles still commonly found in some homes of older construction. Class II supplies provide users with protection from electric shock by enclosing all energized parts with a minimum of two layers of insulation. In addition, IEC 60601-1 requires the output of the power supply to be electrically isolated from the supply mains.

In the example of FIG. 15B, an AC-DC power supply unit converts line power, e.g., 100V, 120 V, 230V, or 240V AC to a regulated 54 volt DC, shown as a battery in the example of 15B. The example power supply may supply up to five or more amps with convection cooling or nine amps with fan cooling.

To provide a bipolar ±27 volt power supply for the driver circuit, a resistive voltage divider with an OPA541 operational amplifier is used to produce a virtual ground. The OPA541 has a rated continuous output current of 5 amps with a peak output current of up to 10 amps. As noted above, other components, arrangements and operating parameters may be desirable depending on the specific implementation of the wireless power transfer system.

The techniques of this disclosure may also be described by the following examples.

Example 1: A system including a power transmitting antenna comprising a coiled conductor defined by a first axis and a second axis perpendicular to the first axis, wherein a single plane comprises the first axis and the second axis; a support layer comprising: a substantially planar top surface and a substantially planar bottom surface opposite the substantially planar top surface arranged parallel to the plane; and a material with a predetermined resiliency; wherein the support layer is configured to support a mass of a user and maintain a predetermined spacing between the plane of the power transmitting antenna and the user during compression of the material from the mass of the user.

Example 2: The system of example 1, wherein the support layer comprises a first layer of a first material with a first resiliency and a second layer of a second material with a second resiliency.

Example 3: The system of examples 1 and 2, wherein the first resiliency is the substantially the same as the second resiliency.

Example 4: The system of any combination of examples 1-3, further includes comprising substantially planar top surface and a substantially planar bottom surface opposite the substantially planar top surface arranged parallel to the plane; and arranged parallel to the plane, such that the power transmitting antenna is sandwiched between the base layer and the support layer.

Example 5: The system of any combination of examples 1-4, wherein the support layer comprises a polyurethane foam.

Example 6: The system of any combination of examples 1-5, wherein the power transmitting antenna is configured to: connect to a driver circuit; generate an electromagnetic field based on a signal received from the driver circuit; and transmit power to a power receiving unit within the electromagnetic field.

Example 7: The system of any combination of examples 1-6, wherein the power transmitting antenna is further configured to send and receive digital communication messages to the power receiving unit.

Example 8: The system of any combination of examples 1-7, wherein the coiled conductor is a first coiled conductor, the power transmitting antenna includes arranged substantially parallel to the plane; electrically isolated from the first coiled conductor; and arranged such that a first center point of the first coiled conductor is at a different location from a second center point of the second coiled conductor.

Example 9: The system of any combination of examples 1-8, wherein the power transmitting antenna first coiled conductor comprises conductors wrapped in both directions.

Example 10: The system of any combination of examples 1-9, wherein the predetermined spacing is a first distance and an output power from the power transmitting antenna is a first output power, and the system further configured to include a second support layer such that the predetermined spacing is a second distance greater than the first distance and the output power from the power transmitting antenna is a second output power greater than the first output power.

Example 11: The system of any combination of examples 1-9, in which a shape of the power transmitting antenna is a square.

Example 12: The system of any of examples 1 through 11, further comprising a power receiving unit within the electromagnetic field, wherein the power transmitting antenna is configured to transmit power to the power receiving unit, wherein the power receiving unit is an implantable medical device.

Example 13: The system of any of examples 1 through 12, wherein the coiled conductor is a first coiled conductor, the system further includes a second coiled conductor defined by a third axis and a fourth axis perpendicular to the third axis, the third axis and the fourth axis defining a second plane; and drive circuitry configured to: generate a first magnetic field in the first coiled conductor with a first drive signal; generate a second magnetic field in the second coiled conductor with a second drive signal, wherein the second drive signal has a phase difference from the first drive signal and an amplitude difference from the first drive signal; adjust an angle of a combined magnetic field comprising the first electromagnetic field and the second magnetic field based on the phase difference and the amplitude difference, wherein the angle is relative to the first plane.

Example 14: A device including a power transmitting antenna comprising a first coiled conductor defined by a first axis and a second axis perpendicular to the first axis, the first axis and the second axis defining a first plane; and a second coiled conductor defined by a third axis and a fourth axis perpendicular to the third axis, the third axis and the fourth axis defining a second plane; and drive circuitry configured to: generate a first magnetic field in the first coiled conductor with a first drive signal; generate a second magnetic field in the second coiled conductor with a second drive signal, wherein the second drive signal has a phase difference from the first drive signal and an amplitude difference from the first drive signal; adjust an angle of a combined magnetic field comprising the first electromagnetic field and the second magnetic field based on the phase difference and the amplitude difference, wherein the angle is relative to the first plane.

Example 15: The device of example 14, wherein the phase difference is zero.

Example 16: The device of examples 14 and 15, wherein the amplitude difference is zero.

Example 17: The device of any combination of examples 14-16, wherein the first plane is coplanar to the second plane.

Example 18: The device of any combination of examples 14-17, wherein the angle of the combined magnetic field is perpendicular to the first plane.

Example 19: The device of any combination of examples 14-18, further includes comprising substantially planar top surface and a substantially planar bottom surface opposite the substantially planar top surface arranged parallel to the first plane; comprising a material with a predetermined resiliency; and configured to receive a load of a user.

Example 20: The device of any combination of examples 14-19, wherein the phase difference and the amplitude difference is a first phase difference and a first amplitude difference, the device further includes has a phase difference from the first drive signal and the second drive signal, and has an amplitude difference from the first drive signal and the second drive signal; and adjust an angle of the combined magnetic field comprising the first electromagnetic field and the second magnetic field based on the first phase difference, the first amplitude difference, the second phase difference, and a second amplitude difference.

Example 21: The device of any combination of examples 14-20, wherein the drive circuitry comprises: a first drive circuit configured to generate the first drive signal, and a second drive circuit configured to generate the first drive signal.

Example 22: A system includes a power transmitting antenna comprising a first coiled conductor defined by a first axis and a second axis perpendicular to the first axis, the first axis and the second axis defining a first plane; and a second coiled conductor defined by a third axis and a fourth axis perpendicular to the third axis, the third axis and the fourth axis defining a second plane; a drive circuit configured to: generate a first magnetic field in the first coiled conductor with a first drive signal; generate a second magnetic field in the second coiled conductor with a second drive signal, wherein the second drive signal has a phase difference from the first drive signal and an amplitude difference from the first drive signal, and adjust an angle of a combined magnetic field comprising the first electromagnetic field and the second magnetic field based on the phase difference and the amplitude difference, wherein the angle is relative to the first plane; and processing circuitry operatively coupled to a memory and to the drive circuit, the processing circuitry configured to control the operation of the drive circuit.

Example 23: The system of example 22, further includes send and receive digital messages from a power receiving device; receive an indication from the power receiving device of a power level received from the combined magnetic field; and cause the driving circuitry to adjust the angle of the combined magnetic field based on the indication of the power level to the power receiving device.

Example 24: The system of examples 22 and 23, wherein responsive to receiving a first indication that the power received is a first power level, the processing circuitry is configured to cause the driving circuitry to adjust the angle such that the processing receives an indication that the power level is a second power level, wherein the second power level is greater than the first power level.

Example 25: The system of any combination of examples 22-24, further includes a substantially planar top surface and a substantially planar bottom surface opposite the substantially planar top surface arranged parallel to the first plane; and a material with a predetermined resiliency, wherein the support layer is configured to receive a load of a user; and wherein the processing is configured to, responsive to determining the load of the user caused a change of the angle of the combined magnetic field, cause the driving circuitry to adjust the angle of the combined magnetic field to compensate for the change of the angle caused by the load of the user.

Example 26: A power transmitting antenna, the antenna comprising a coiled conductor defined by a first axis and a second axis perpendicular to the first axis, wherein the antenna defines an outside edge and an inside edge of a coil, wherein the outside edge defines an outside dimension and the inside edge defines an inside dimension; and wherein a single plane comprises the first axis and the second axis, the coiled conductor comprising: a first portion of the coiled conductor wrapped with a plurality of turns in a clockwise direction from the outside dimension to the inside dimension; a second portion of the coiled conductor wrapped with a plurality of turns in a counter-clockwise direction from the inside dimension to the outside dimension, wherein the first portion is electrically connected to the second portion.

Example 27: The power transmitting antenna of example 26, wherein the first portion is electrically connected to the second portion at the inside dimension.

Example 28: The power transmitting antenna of any of examples 26 and 27, wherein: the antenna is configured to receive a drive signal; the drive signal generates an electromagnetic field in the coiled conductor; a first electric field of the electromagnetic field generated by the first portion at least partially cancels a second electric field of the electromagnetic field generated by the second portion; and a first magnetic field of the electromagnetic field generated by the first portion adds to a second magnetic field of the electromagnetic field generated by the second portion.

Example 29: The power transmitting antenna of any of examples 26 through 28, wherein a first conductor of an inward wrapped coil portion is proximal to a last conductor of an outward wrapped coil portion.

Example 30: A method comprising controlling, by processing circuitry operatively coupled to a memory, a drive circuit to activate a first coil of a power transmitting antenna: receiving, by the processing circuitry, an indication from a power receiving unit of a first amount of power received from the power transmitting antenna; storing, by the processing circuitry at a memory location of the memory, a first value associated with the first amount of power; controlling, by the processing circuitry, the drive circuit to deactivate the first coil; controlling, by the processing circuitry, the drive circuit to activate a second coil of the power transmitting antenna; receiving, by the processing circuitry, an indication from the power receiving unit of a second amount of power received from the power transmitting antenna; storing, by the processing circuitry at a memory location of the memory, a second value associated with the second amount of power; controlling, by the processing circuitry, the drive circuit to deactivate the second coil; comparing, by the processing circuitry, the first value to the second value, and controlling, by the processing circuitry and based on the comparison and responsive to the first value being greater than the second value, the drive circuit to deliver power to the power receiving unit by activating the first coil.

Example 31: The method of example 30, wherein a medical device comprises the power receiving unit.

Example 32: The method of any of examples 30 and 31, wherein an implantable medical device comprises the power receiving unit.

Various examples of the disclosure have been described. These and other examples are within the scope of the following claims.

1. A system comprising:
a power transmitting antenna comprising a coiled conductor defined by a first axis and a second axis perpendicular to the first axis, wherein a single plane comprises the first axis and the second axis;
a support layer comprising:
a substantially planar top surface and a substantially planar bottom surface opposite the substantially planar top surface arranged parallel to the plane; and
a material with a predetermined resiliency; wherein the support layer is configured to support a mass of a user and maintain a predetermined spacing between the plane of the power transmitting antenna and the user during compression of the material from the mass of the user.

2. The system of claim 1, wherein the support layer comprises a first layer of a first material with a first resiliency and a second layer of a second material with a second resiliency.

3. The system of claim 2, wherein the first resiliency is the substantially the same as the second resiliency.

4. The system of claim 1, further comprising a base layer, the base layer:
comprising substantially planar top surface and a substantially planar bottom surface opposite the substantially planar top surface arranged parallel to the plane; and
arranged parallel to the plane, such that the power transmitting antenna is sandwiched between the base layer and the support layer.

5. The system of claim 1, wherein the support layer comprises a polyurethane foam.

6. The system of claim 1, wherein the power transmitting antenna is configured to:
connect to a driver circuit;
generate an electromagnetic field based on a signal received from the driver circuit; and
transmit power to a power receiving unit within the electromagnetic field.

7. The system of claim 1, wherein the power transmitting antenna is further configured to send and receive digital communication messages to the power receiving unit.

8. The system of claim 1, wherein the coiled conductor is a first coiled conductor, the power transmitting antenna comprising a second coiled conductor:
arranged substantially parallel to the plane;
electrically isolated from the first coiled conductor; and
arranged such that a first center point of the first coiled conductor is at a different location from a second center point of the second coiled conductor.

9. The system of claim 8, wherein the first coiled conductor of the power transmitting antenna comprises conductors wrapped in both directions.

10. The system of claim 1,
wherein the predetermined spacing is a first distance and an output power from the power transmitting antenna is a first output power and the support layer is a first support layer, and
the system further configured to include a second support layer such that the predetermined spacing is a second distance greater than the first distance and the output power from the power transmitting antenna is a second output power greater than the first output power.

11. The system of claim 1, in which a shape of the power transmitting antenna is a square.

12. The system of claim 1, further comprising a power receiving unit within the electromagnetic field, wherein the power transmitting antenna is configured to transmit power to the power receiving unit, wherein the power receiving unit is an implantable medical device.

13. The system of claim 1, wherein the coiled conductor is a first coiled conductor, the system further comprising:
a second coiled conductor defined by a third axis and a fourth axis perpendicular to the third axis, the third axis and the fourth axis defining a second plane; and
drive circuitry configured to:
generate a first magnetic field in the first coiled conductor with a first drive signal;
generate a second magnetic field in the second coiled conductor with a second drive signal, wherein the second drive signal has a phase difference from the first drive signal and an amplitude difference from the first drive signal;
adjust an angle of a combined magnetic field comprising the first electromagnetic field and the second magnetic field based on the phase difference and the amplitude difference, wherein the angle is relative to the first plane.

14. A power transmitting antenna comprising:
the antenna comprising a coiled conductor defined by a first axis and a second axis perpendicular to the first axis,
wherein the antenna defines an outside edge and an inside edge of a coil,
wherein the outside edge defines an outside dimension and the inside edge defines an inside dimension; and
wherein a single plane comprises the first axis and the second axis,
the coiled conductor comprising:
a first portion of the coiled conductor wrapped with a plurality of turns in a clockwise direction from the outside dimension to the inside dimension;
a second portion of the coiled conductor wrapped with a plurality of turns in a counter-clockwise direction from the inside dimension to the outside dimension, wherein the first portion is electrically connected to the second portion.

15. A method comprising:
controlling, by processing circuitry operatively coupled to a memory, a drive circuit to activate a first coil of a power transmitting antenna;
receiving, by the processing circuitry, an indication, from a power receiving unit, of a first amount of power received from the power transmitting antenna;
storing, by the processing circuitry at the memory, a first value associated with the first amount of power;
controlling, by the processing circuitry, the drive circuit to deactivate the first coil;
controlling, by the processing circuitry, the drive circuit to activate a second coil of the power transmitting antenna;
receiving, by the processing circuitry, an indication, from the power receiving unit, of a second amount of power received from the power transmitting antenna;
storing, by the processing circuitry at the memory, a second value associated with the second amount of power;
controlling, by the processing circuitry, the drive circuit to deactivate the second coil;
comparing, by the processing circuitry, the first value to the second value; and
controlling, by the processing circuitry and based on the comparison and responsive to the first value being greater than the second value, the drive circuit to deliver power to the power receiving unit by activating the first coil.

16. The method of claim 15, wherein the first coil of the power transmitting antenna comprises:
a coiled conductor defined by a first axis and a second axis perpendicular to the first axis, wherein a single plane comprises the first axis and the second axis;
a support layer comprising:
a substantially planar top surface and a substantially planar bottom surface opposite the substantially planar top surface arranged parallel to the plane; and
a material with a predetermined resiliency; wherein the support layer is configured to support a mass of a user and maintain a predetermined spacing between the plane of the power transmitting antenna and the user during compression of the material from the mass of the user.

17. The method of claim 16,
wherein the predetermined spacing is a first distance and an output power from the power transmitting antenna is a first output power and the support layer is a first support layer, and
the system further configured to include a second support layer such that the predetermined spacing is a second distance greater than the first distance and the output power from the power transmitting antenna is a second output power greater than the first output power.

18. The method of claim 16, wherein the second coil comprises a second coiled conductor:
arranged substantially parallel to the plane;
electrically isolated from the first coiled conductor; and
arranged such that a first center point of the first coiled conductor is at a different location from a second center point of the second coiled conductor.

19. The method of claim 15, wherein the first coil and the second coil of the power transmitting antenna each comprise conductors wrapped in both directions.

20. The method of claim 15, further comprising sending digital communication messages to, and receiving digital communication messages from, the power receiving unit via the power transmitting antenna.
